(19) United States
(12) Patent Application Publication
(10) Pub. No.: US 2018/0028472 A1
(43) Pub. Date: Feb. 1, 2018

(54) TAMPER-RESISTANT DOSAGE FORM COMPRISING A POLYETHYLENE GLYCOL GRAFT COPOLYMER

(71) Applicant: GRÜNENTHAL GMBH, Aachen (DE)

(72) Inventor: Lutz BARNSCHEID, Mönchengladbach (DE)

(73) Assignee: GRÜNENTHAL GMBH, Aachen (DE)

(21) Appl. No.: 15/665,969

(22) Filed: Aug. 1, 2017

Related U.S. Application Data
(63) Continuation of application No. PCT/EP2016/052046, filed on Feb. 1, 2016.

(30) Foreign Application Priority Data
Feb. 3, 2015 (EP) 15153679.4

(51) Int. Cl. A61K 31/135; A61K 9/20

(52) U.S. Cl. CPC A61K 31/135; A61K 9/2031; A61K 9/2095

(57) ABSTRACT

A tamper-resistant, oral pharmaceutical dosage form comprising a pharmacologically active ingredient having psychotropic action and a polyethylene glycol graft copolymer, wherein the content of the polyethylene glycol graft copolymer is at least 25 wt.-%, relative to the total weight of the pharmaceutical dosage form. Also disclosed is the manufacture of the dosage form and its use.

This application is a continuation of International Patent Application No. PCT/EP2016/052046, filed Feb. 1, 2016, which, in turn, claims priority of European Patent Application No. EP 15153679.4, filed Feb. 3, 2015, the entire contents of which patent applications are hereby incorporated by reference herein.

The invention relates to a tamper-resistant, oral pharmaceutical dosage form comprising a pharmacologically active ingredient having psychotropic action and a polyethylene glycol graft copolymer, wherein the content of the polyethylene glycol graft copolymer is at least 25 wt.-%, relative to the total weight of the pharmaceutical dosage form. Preferably, the dosage form according to the invention provides prolonged release of the pharmacologically active ingredient that follows zero order kinetics.

A large number of drugs have a potential for being abused or misused, i.e. they can be used to produce effects which are not consistent with their intended use. Thus, e.g. opioids which exhibit an excellent efficacy in controlling severe to extremely severe pain are frequently abused to induce euphoric states similar to being intoxicated. In particular, drugs having a psychotropic effect are abused accordingly.

To enable abuse, the corresponding pharmaceutical dosage forms, such as pharmaceutical dosage forms or capsules are crushed, for example ground by the abuser, the drug is extracted from the thus obtained powder using a preferably aqueous liquid and after being optionally filtered through cotton wool or cellulose wadding, the resultant solution is administered parenterally, in particular intravenously. This type of administration results in an even faster diffusion of the drug compared to the oral abuse, with the result desired by the abuser, namely the kick. This kick or these intoxication-like, euphoric states are also reached if the powdered pharmaceutical dosage form is administered nasally, i.e. is sniffed.

Various concepts for the avoidance of drug abuse have been developed.

It has been proposed to incorporate in pharmaceutical dosage forms aversive agents and/or antagonists in a manner so that they only produce their aversive and/or antagonizing effects when the pharmaceutical dosage forms are tampered with. However, the presence of such aversive agents is principally not desirable and there is a need to provide sufficient tamper-resistance without relying on aversive agents and/or antagonists.

Another concept to prevent abuse relies on the mechanical properties of the pharmaceutical dosage forms, particularly an increased breaking strength (resistance to crushing). The major advantage of such pharmaceutical dosage forms is that comminuting, particularly pulverization, by conventional means, such as grinding in a mortar or fracturing by means of a hammer, is impossible or at least substantially impeded. Thus, the pulverization, necessary for abuse, of the pharmaceutical dosage forms by the means usually available to a potential abuser is prevented or at least complicated. Such pharmaceutical dosage forms are useful for avoiding drug abuse of the pharmacologically active ingredient contained therein, as they may not be powdered by conventional means and thus, cannot be administered in powdered form, e.g. nasally. The mechanical properties, particularly the high breaking strength of these pharmaceutical dosage forms renders them tamper-resistant. In the context of such tamper-resistant pharmaceutical dosage forms it can be referred to, e.g., WO 2005/016313, WO 2005/016314, WO 2005/063214, WO 2005/102286, WO 2006/002883, WO 2006/002884, WO 2006/002886, WO 2006/082097, WO 2006/082099, and WO 2009/092601.

The properties of these pharmaceutical dosage forms of the prior art, however, are not satisfactory in every respect. In particular, the tamper resistant dosage forms of the prior art that provide prolonged release of the drug typically show a release profile following first order kinetics or second order kinetics, i.e. the relative amount of drug that is release per time unit changes over time.

There is a demand for tamper resistant pharmaceutical dosage forms that provide substantially linear drug release, i.e. zero order kinetics.

Retard tablets with substantially linear drug release are known from the prior art. However, the technical concepts to achieve substantially linear drug release are comparatively laborious and thus expensive. Furthermore, they are usually not compatible with the concepts for achieving tamper resistance. In OROS technology, the dosage forms comprise various layers having different function such that one layer is osmotically active whereas another layer contains the drug (cf. e.g. U.S. Pat. No. 5,460,826, U.S. Pat. No. 6,245,357). Other concepts aim at keeping the diffusion area constant during the entire release process e.g. by providing the dosage forms with a water-resistant coating or with a specific shape.

US 2012/231083 relates to a medicament which results in delivery of a therapeutic level of one or more cannabinoids during a clinically relevant therapeutic window. The therapeutic window is a longer window than provided by an immediate release medicament containing an equivalent amount of the cannabinoid. Oral administration of the compositions provides therapeutic dosing while maintaining safe, side effect sparing, levels of a cannabinoid.

U.S. Pat. No. 5,082,668 discloses an osmotically driven dosage form, namely a device comprising a wall that surrounds a compartment. The compartment comprises a beneficial agent composition and a push composition. A passageway in the wall connects the compartment with the exterior of the device for delivering the beneficial agent at a rate governed, in combination, by the wall, the beneficial agent composition and the push composition through the passageway of the device over time.

U.S. Pat. No. 7,300,668 relates to a dosage form comprising: a three-dimensionally printed innermost region comprising a first regional concentration of at least one active pharmaceutical ingredient; and plural three-dimensionally printed non-innermost regions in nested arrangement and comprising: a) one or more nested internal regions, wherein an internal region completely surrounds and is in contact with the innermost regions, and any other internal region present completely surrounds another internal region located to the interior thereof; and b) an outermost region completely surrounding an internal region, wherein the internal and outermost regions are in nested arrangement, wherein the at least one active pharmaceutical ingredient is released in approximately a zero-order release.

WO 03/024426 discloses a controlled release pharmaceutical composition for oral use comprising a solid dispersion of: i) at least one therapeutically, prophylactically and/or diagnostically active substance, which at least partially is in an amorphous form, ii) a pharmaceutically acceptable polymer that has plasticizing properties, and iii) optionally, a stabilizing agent, the at least one active substance having a limited water solubility, and the composition being designed to release the active substance with a substantially zero order release. Zero order release is provided by a coating that remains intact during the release phase and covers the matrix composition in such a manner that only a specific surface area is subject to erosion. Thereby the surface area from which the active substance is released is kept substantially constant during the time period.

WO 2008/086804 discloses abuse resistant polyglycol-based pharmaceutical compositions. The composition contains one or more polyglycols and one or more active substances and it is resistant to crushing, melting and/or extraction. Moreover, such compositions have the same or lower solubility in ethanolic-aqueous medium, i.e. they are not subject to ethanol-induced dose dumping effect.

WO 2008/148798 discloses a layered pharmaceutical composition suitable for oral use in the treatment of diseases where absorption takes place over a large part of the gastrointestinal tract.

WO 2010/057036 discloses a solid composition and methods for making and using the solid composition are provided. The solid composition comprises: (a) at least one active agent with a solubility of less than 0.3 mg/ml in an aqueous solution with a pH of at most 6.8 at a temperature of 37° C.; and (b) a hydrophilic polymer matrix composition comprising: i) a hydrophilic polymer selected from the group consisting of METHOCEL®, POLYOX® WSR 1105 and combinations thereof; and optionally ii) a hydrophobic polymer selected from the group consisting of Ethocel 20 premium; and (c) an alkalizer selected from the group consisting of calcium carbonate, magnesium oxide heavy and sodium bicarbonate; wherein the composition provides at least 70% release of the active between 7 to 12 hours following oral administration.

WO 2012/028319 relates to a pharmaceutical dosage form exhibiting a breaking strength of at least 500 N, said dosage form containing a pharmacologically active ingredient; an inorganic salt (B); and a polyalkylene oxide (C) having a weight average molecular weight of at least 200,000 g/mol, wherein the content of the polyalkylene oxide (C) is at least 20 wt.-%, based on the total weight of the dosage form; wherein the pharmacologically active ingredient is present in a controlled-release matrix comprising the inorganic salt (B) and the polyalkylene oxide (C) and wherein, under in vitro conditions, the release profile of the pharmacologically active ingredient from said matrix comprises at least a time interval during which the release follows zero order kinetics.

WO 2014/059512 discloses a formulation that comprises at least one active substance and at least one coat comprising Eudragit E (dimethylaminoethyl methacrylate copolymer), wherein the formulation is free of any active substance external to the coat. The formulation is effective in preventing significant dose dumping in alcoholic/non-alcoholic beverage(s). In another aspect, a formulation is provided that comprises a loading dose having at least one active substance, wherein the release of the at least one active substance shows a Point Of Divergence (POD), in a dissolution profile, with the loading dose representing a point in a timeline where the history of the dissolution or release rate changes from an onset of action to another set of points in the timeline represented by a controlled release. The formulation may be used for releasing up to about 55% of a total dose as a loading dose in order to manage pain.

V. Pillay et al., Journal of Controlled Release, 67 (2000) 67-78 discloses an approach for constant rate delivery of highly soluble bioactives from a simple monolithic system prepared by direct compression at ambient conditions.

M. E. McNeill et al., J Biomater Sci Polym 1996, 7(11), 953-63 relates to properties controlling the diffusion and release of water-soluble solutes from poly(ethylene oxide) hydrogels. Part 4 deals with extended constant rate release from partly-coated spheres.

D. Henrist et al., J. Controlled Release, 75 (2001) 391-400 relates to in vitro and in vivo evaluation of starch-based hot stage extruded double matrix systems. The objective of developing a double matrix system consisting of a hot stage extruded starch pipe surrounding a hot stage extruded and drug-containing starch core, was to obtain a monolithic matrix system applicable in the domain of sustained drug release. The behavior of the systems was evaluated through dissolution testing and through a randomized crossover bioavailability study on nine male volunteers. All double matrix systems showed in vitro a nearly constant drug release profile after an initial slower release phase of 4 h. This initial slower release phase was avoided by loading the starch pipe with a small amount of drug.

L. Yang et al., J. Pharm. Sciences, 85(2), 1996, 170-173 relates to zero-order release kinetics from a self-correcting floatable asymmetric configuration drug delivery system.

It is an object of the invention to provide tamper-resistant, oral pharmaceutical dosage forms containing a pharmacologically active ingredient having psychotropic action which have advantages compared to the pharmaceutical dosage forms of the prior art.

This object has been achieved by the patent claims.

It has been surprisingly found that a pharmaceutical dosage form comprising a polyethylene glycol graft copolymer and a pharmacologically active ingredient having psychotropic action can be prepared, wherein the pharmaceutical dosage form exhibits tamper resistance and provides substantially linear drug release based upon its composition, particularly of the matrix in which the pharmacologically active ingredient having psychotropic action is preferably embedded. Thus, laborious multilayered structures, water-resistant coatings and the like can be avoided. Furthermore, the risk of unintentional overdose as a consequence of damage of the release system (dose-dumping) can be substantially reduced or even completely be suppressed.

### BRIEF DESCRIPTION OF THE DRAWINGS

The invention will now be described in greater detail with reference to the drawings, wherein:

FIG. 1 is a load-displacement diagram showing the result of the breaking strength test of Example 1; and

FIG. 2 is a graph depicting the in vitro dissolution profiles of an embodiment of the present invention compared to a comparative example.

A first aspect of the invention relates to a tamper-resistant, oral pharmaceutical dosage form comprising a pharmacologically active ingredient having psychotropic action and a polyethylene glycol graft copolymer, which dosage form preferably provides resistance against solvent extraction, and/or resistance against grinding, and/or resistance against dose-dumping in aqueous ethanol.

Preferably, the pharmaceutical dosage form according to the invention is thermoformed, more preferably hot-melt extruded. Thermoforming preferably means that in the course of the manufacture of the pharmaceutical dosage form the mixture comprising the polyethylene glycol graft copolymer and the pharmacologically active ingredient is heated to a temperature above ambient temperature, preferably at least 60° C. or at least 80° C., and compressed, preferably at pressures of at least 1 bar or at least 1.5 bar. The compression force may be exerted prior to, during or subsequent to the application of heat.

As used herein, the term “pharmaceutical dosage form” refers to a pharmaceutical entity that is comprised of a pharmacologically active ingredient and which is actually administered to, or taken by, a patient. It may be compressed or molded in its manufacture, and it may be of almost any size, shape, weight, and color.

The pharmaceutical dosage form is preferably solid or semisolid.

Examples of pharmaceutical dosage forms according to the invention include, but are not limited to, tablets, capsules, pills, granules, pellets, sachets and effervescent, powders, and the like. In an embodiment of the present invention, the composition is formulated in a capsule. In accordance with this embodiment, the pharmaceutical dosage form comprises a hard or soft gelatin capsule.

Most pharmaceutical dosage forms are intended to be swallowed whole and accordingly, the pharmaceutical dosage forms according to the invention are designed for oral administration.

In a preferred embodiment, the pharmaceutical dosage form according to the invention is monolithic. In this regard, monolithic preferably means that the pharmaceutical dosage form is formed or composed of material without joints or seams or consists of or constitutes a single unit.

In another preferred embodiment, the pharmaceutical dosage form according to the invention is not monolithic. Preferably, the pharmaceutical dosage form according to the invention is multiparticulate, i.e. comprises a multitude of particles. An advantage of multiparticulate pharmaceutical dosage forms is that the particles may be mixed in different amounts to thereby produce pharmaceutical dosage forms of different strengths.

In a preferred embodiment, the pharmaceutical dosage form according to the invention can be regarded as a MUPS formulation (multiple unit pellet system). Preferably, the pharmaceutical dosage form according to the invention contains all ingredients in a dense compact unit which in comparison to capsules has a comparatively high density. Under these circumstances, the pharmaceutical dosage forms according to the invention preferably comprise subunits having different morphology and properties, namely drug-containing particles and an outer matrix material, wherein the particles form a discontinuous phase within the outer matrix material. The constituents of the outer matrix material are preferably different from the constituents of the drug-containing particles. Preferably, the outer matrix material neither contains a pharmacologically active ingredient having psychotropic action nor a polyethylene glycol graft copolymer.

The particles typically have mechanical properties that differ from the mechanical properties of the outer matrix material. Preferably, the particles have a higher mechanical strength than the outer matrix material. The particles can preferably be visualized by conventional means such as solid state nuclear magnetic resonance spectroscopy, raster electron microscopy, terahertz spectroscopy and the like.

The pharmaceutical dosage form according to the invention has preferably a total weight in the range of 0.01 to 1.5 g, more preferably in the range of 0.05 to 1.2 g, still more preferably in the range of 0.1 g to 1.0 g, yet more preferably in the range of 0.2 g to 0.9 g, and most preferably in the range of 0.3 g to 0.8 g. In a preferred embodiment, the total weight of the pharmaceutical dosage form is within the range of 350±300 mg, more preferably 350±250 mg, still more preferably 350±200 mg, yet more preferably 350±150 mg, most preferably 350±100 mg, and in particular 350±50 mg. In another preferred embodiment, the total weight of the pharmaceutical dosage form is within the range of 500±450 mg, more preferably 500±300 mg, still more preferably 500±200 mg, yet more preferably 500±150 mg, most preferably 500±100 mg, and in particular 500±50 mg.

In a preferred embodiment, the pharmaceutical dosage form according to the invention is a round pharmaceutical dosage form. Pharmaceutical dosage forms of this embodiment preferably have a diameter in the range of 1 mm to 30 mm, in particular in the range of 2 mm to 25 mm, more in particular 5 mm to 23 mm, even more in particular 7 mm to 13 mm; and a thickness in the range of 1.0 mm to 12 mm, in particular in the range of 2.0 mm to 10 mm, even more in particular from 3.0 mm to 9.0 mm, even further in particular from 4.0 mm to 8.0 mm.

In another preferred embodiment, the pharmaceutical dosage form according to the invention is an oblong pharmaceutical dosage form. Pharmaceutical dosage forms of this embodiment preferably have a lengthwise extension (longitudinal extension) of 1 mm to 30 mm, in particular in the range of 2 mm to 25 mm, more in particular 5 mm to 23 mm, even more in particular 7 mm to 20 mm; a width in the range of 1 mm to 30 mm, in particular in the range of 2 mm to 25 mm, more in particular 5 mm to 23 mm, even more in particular 7 mm to 13 mm; and a thickness in the range of 1.0 mm to 12 mm, in particular in the range of 2.0 mm to 10 mm, even more in particular from 3.0 mm to 9.0 mm, even further in particular from 4.0 mm to 8.0 mm.

When the pharmaceutical dosage form according to the invention is monolithic, it preferably has an extension in any direction of at least 2.0 mm, more preferably at least 2.5 mm, still more preferably at least 3.0 mm, yet more preferably at least 3.5 mm, even more preferably at least 4.0 mm, most preferably at least 4.5 mm and in particular at least 5.0 mm. Preferably, when the dosage form is monolithic, it has an extension in any direction of more than 2.0 mm.

For the purpose of specification, “in any direction” preferably means in every direction in the three-dimensional space.

The pharmaceutical dosage form according to the invention may optionally comprise a coating, e.g. a cosmetic coating. The coating is preferably applied after formation of the pharmaceutical dosage form. The coating may be applied prior to or after the curing process. The pharmaceutical dosage forms according to the invention are preferably film coated with conventional film coating compositions. Suitable coating materials are commercially available, e.g. under the trademarks Opadry® and Eudragit®.

Examples of suitable materials include cellulose esters and cellulose ethers, such as methylcellulose (MC), hydroxypropylmethylcellulose (HPMC), hydroxypropylcellulose (HPC), hydroxyethylcellulose (HEC), sodium carboxymethylcellulose (Na-CMC), poly(meth)acrylates, such as aminoalkylmethacrylate copolymers, methacrylic acid methylmethacrylate copolymers, methacrylic acid methylmethacrylate copolymers; vinyl polymers, such as polyvinylpyrrolidone, polyvinyl caprolactam, polyvinylacetate; and natural film formers.

The coating can be resistant to gastric juices and dissolve as a function of the pH value of the release environment. By means of this coating, it is possible to ensure that the pharmaceutical dosage form according to the invention passes through the stomach undissolved and the active compound is only released in the intestines. The coating which is resistant to gastric juices preferably dissolves at a pH value of between 5 and 7.5.

The coating can also be applied e.g. to improve the aesthetic impression and/or the taste of the pharmaceutical dosage forms and the ease with which they can be swallowed. Coating the pharmaceutical dosage forms according to the invention can also serve other purposes, e.g. improving stability and shelf-life. Suitable coating formulations comprise a film forming polymer such as, for example, hydroxypropyl methylcellulose, e.g. hypromellose, a plasticizer such as, for example, a glycol, e.g. propylene glycol or polyethylene glycol, an opacifier, such as, for example, titanium dioxide, and a film smoothener, such as, for example, talc. Suitable coating solvents are water as well as organic solvents. Examples of organic solvents are alcohols, e.g. ethanol or isopropanol, ketones, e.g. acetone, or halogenated hydrocarbons, e.g. methylene chloride. Coated pharmaceutical dosage forms according to the invention are preferably prepared by first making the cores and subsequently coating said cores using conventional techniques, such as coating in a coating pan.

Preferably, the pharmaceutical dosage form according to the invention comprises a prolonged release matrix. The prolonged release matrix in turn preferably comprises the polyethylene glycol graft copolymer as prolonged release matrix material and optionally additional prolonged release matrix material.

In a preferred embodiment, the prolonged release matrix does not contain any additional prolonged release matrix material.

The pharmacologically active ingredient is preferably embedded in the prolonged release matrix comprising the polyethylene glycol graft copolymer. Preferably, the pharmacologically active ingredient is dispersed in the prolonged release matrix.

Preferably, the pharmaceutical dosage form provides prolonged release of the pharmacologically active ingredient. Particularly preferably, the prolonged release matrix comprising the polyethylene glycol graft copolymer provides prolonged release of the pharmacologically active ingredient embedded therein.

In a preferred embodiment,
(i) the pharmacologically active ingredient is embedded in a prolonged release matrix comprising the polyethylene glycol graft copolymer; and/or
(ii) the pharmaceutical dosage form provides prolonged release of the pharmacologically active ingredient.

When the pharmaceutical dosage form according to the invention is monolithic and comprises a prolonged release matrix, the prolonged release matrix preferably forms the body of the pharmaceutical dosage form.

When the pharmaceutical dosage form according to the invention is multiparticulate, e.g. in form of pellets, the particles preferably comprise the prolonged release matrix and at least a portion of the total amount of the pharmacologically active ingredient that is contained in the pharmaceutical dosage form. Preferably, the particles comprise the total amount of the pharmacologically active ingredient that is contained in the pharmaceutical dosage form.

When the pharmaceutical dosage form according to the invention can be regarded as a MUPS formulation which preferably comprises drug-containing particles and an outer matrix material, the outer matrix material is not a constituent of the prolonged release matrix and is to be distinguished from the prolonged release matrix material and the optionally present additional prolonged release matrix material of the prolonged release matrix of the pharmaceutical dosage form according to the invention.

For the purpose of specification, the term “particle” refers to a discrete mass of material that is solid, e.g. at 20° C. or at room temperature or ambient temperature. Preferably a particle is solid at 20° C. Preferably, the particles are monoliths. Preferably, the pharmacologically active ingredient and the polyethylene glycol graft copolymer are intimately homogeneously distributed in the particles so that the particles do not contain any segments where either pharmacologically active ingredient is present in the absence of polyethylene glycol graft copolymer or where polyethylene glycol graft copolymer is present in the absence of pharmacologically active ingredient. Typically, segments in this regard are of macroscopic size, e.g. spheres having a radius of 0.1 mm.

When the pharmaceutical dosage form is multiparticulate, it preferably comprises a multitude i.e. plurality of particles containing pharmacologically active ingredient (drug-containing particles) and may optionally further comprise particles not containing any pharmacologically active ingredient (drug-free particles).

In a preferred embodiment, the pharmaceutical dosage form preferably comprises at most 10, more preferably at most 9, still more preferably at most 8, yet more preferably at most 7, even more preferably at most 6, most preferably at most 5, and in particular at most 4 or 3 or 2 drug-containing particles. In another preferred embodiment, the pharmaceutical dosage form preferably comprises at least 2, more preferably at least 4, still more preferably at least 6, yet more preferably at least 8, even more preferably at least 10, most preferably at least 15 and in particular at least 20 or at least 50 or at least 100 drug-containing particles.

When the particles are film coated, the polyethylene glycol graft copolymer is preferably homogeneously distributed in the core of the particles, i.e. the film coating preferably does not contain polyethylene glycol graft copolymer.

When the particles contain a prolonged release matrix and are film coated, the prolonged release matrix is preferably homogeneously distributed in the core of the particles, i.e. the film coating preferably neither contains prolonged release matrix material nor optionally present additional prolonged release matrix material.

The particles are preferably of macroscopic size, typically the average diameter is within the range of from 100 μm to 2000 μm, preferably 200 μm to 1500 μm, more preferably 300 μm to 1500 μm, still more preferably 400 μm to 1500 μm, most preferably 500 μm to 1500 μm, and in particular 600 μm to 1500 μm. Preferably, the particles in the pharmaceutical dosage form have an average particle size of at least 50 μm, more preferably at least 100 μm, still more preferably at least 150 μm or at least 200 μm, yet more preferably at least 250 μm or at least 300 μm, most preferably at least 400 μm or at least 500 μm, and in particular at least 550 μm or at least 600 μm. Preferably, the particles in the pharmaceutical dosage form have an average particle size of at least 700 μm, more preferably at least 800 μm, most preferably at least 900 μm and in particular at least 1000 μm.

In a preferred embodiment, the pharmaceutical dosage forms according to the invention comprise particles as a discontinuous phase, i.e. the particles form a discontinuous phase in an outer matrix material which in turn preferably forms a continuous phase. In this regard, discontinuous means that not each and every particle is in intimate contact with another particle but that the particles are at least partially separated from one another by the outer matrix material in which the particles are embedded. In other words, the particles preferably do not form a single coherent mass within the pharmaceutical dosage forms according to the invention.

Preferably, when the pharmaceutical dosage form is multiparticulate, the content of the particles in the pharmaceutical dosage forms according to the invention is at most 95 wt.-%, more preferably at most 90 wt.-%, still more preferably at most 85 wt.-%, yet more preferably at most 80 wt.-%, most preferably at most 75 wt.-% and in particular at most 70 wt.-%, based on the total weight of the pharmaceutical dosage forms.

Preferably, when the pharmaceutical dosage form is multiparticulate, the content of the particles in the pharmaceutical dosage forms according to the invention is at least 10 wt.-%, at least 15 wt.-%, at least 20 wt.-% or at least 25 wt.-%; more preferably at least 30 wt.-%, at least 35 wt.-%, at least 40 wt.-% or at least 45 wt.-%; most preferably at least 50 wt.-%, at least 55 wt.-%, at least 60 wt.-% or at least 65 wt.-%; and in particular at least 70 wt.-%, at least 75 wt.-%, at least 80 wt.-% or at least 85 wt.-%; based on the total weight of the pharmaceutical dosage form.

When the pharmaceutical dosage form is multiparticulate, the shape of the particles is not particularly limited. As the particles are preferably manufactured by hot-melt extrusion, preferred particles present in the pharmaceutical dosage forms according to the invention are generally cylindrical in shape. The diameter of such particles is therefore the diameter of their circular cross section. The cylindrical shape is caused by the extrusion process according to which the diameter of the circular cross section is a function of the extrusion die and the length of the cylinders is a function of the cutting length according to which the extruded strand of material is cut into pieces of preferably more or less predetermined length.

Typically, the aspect ratio is regarded as an important measure of the spherical shape. The aspect ratio is defined as the ratio of the maximal diameter (dₘₐₓ) and its orthogonal Feret-diameter. For aspherical particles, the aspect ratio has values above 1. The smaller the value the more spherical is the particle. In a preferred embodiment, the aspect ratio of the particles is at most 1.40, more preferably at most 1.35, still more preferably at most 1.30, yet more preferably at most 1.25, even more preferably at most 1.20, most preferably at most 1.15 and in particular at most 1.10. In another preferred embodiment, the aspect ratio of the particles is at least 1.10, more preferably at least 1.15, still more preferably at least 1.20, yet more preferably at least 1.25, even more preferably at least 1.30, most preferably at least 1.35 and in particular at least 1.40.

Preferred particles have an average length and average diameter of 1000 μm or less. When the particles are manufactured by extrusion technology, the “length” of particles is the dimension of the particles that is parallel to the direction of extrusion. The “diameter” of particles is the largest dimension that is perpendicular to the direction of extrusion.

Particularly preferred particles have an average diameter of less than 2000 μm, more preferably less than 1000 or 800 μm, still more preferably of less than 650 μm. Especially preferred particles have an average diameter of less than 700 μm, particularly less than 600 μm, still more particularly less than 500 μm, e.g. less than 400 μm. Particularly preferred particles have an average diameter in the range of 200-1500 μm, more preferably 400-800 μm, still more preferably 450-700 μm, yet more preferably 500-650 μm, e.g. 500-600 μm. Further preferred particles have an average diameter of between 300 μm and 400 μm, of between 400 μm and 500 μm, or of between 500 μm and 600 μm, or of between 600 μm and 700 μm or of between 700 μm and 800 μm.

In a preferred embodiment, particles that are present in the pharmaceutical dosage forms according to the invention have an average length in the range of 500 to 5000 μm, more preferably 750 to 4600 μm, still more preferably 1000 to 4200 μm, yet more preferably 1250 to 3800 μm, even more preferably 1500 to 3400 μm, most preferably 1750 to 3200 μm and in particular 2000 to 3000 μm. According to this embodiment, particles that are present in the pharmaceutical dosage forms according to the invention preferably have an average length of less than 4000 μm, more preferably less than 3000 μm, still more preferably less than 2000 μm, e.g. a length of 1800 μm, 1600 μm, 1400 μm, 1200 μm or 1000 μm. In another preferred embodiment, particles that are present in the pharmaceutical dosage forms according to the invention have an average length in the range of 200 to 1000 μm, more preferably 400 to 800 μm, still more preferably 450 to 700 μm, yet more preferably 500 to 650 μm, e.g. 500 to 600 μm. According to this embodiment, particles that are present in the pharmaceutical dosage forms according to the invention preferably have an average length of less than 1000 μm, more preferably less than 800 μm, still more preferably less than 650 μm, e.g. a length of 800 μm, 700 μm 600 μm, 500 μm, 400 μm or 300 μm. Especially preferred particles have an average length of less than 700 μm, particularly less than 650 μm, still more particularly less than 550 μm, e.g. less than 450 μm. The minimum average length of the particles is determined by the cutting step and may be, e.g. 4.0 mm, 3.0 mm, 2.0 mm, 2.5 mm, 2.0 mm, 1.5 mm, 1.0 mm, 0.9 mm, 0.8 mm, 0.7 mm, 0.6 mm, 0.5 mm, 0.4 mm, 0.3 mm or 0.2 mm.

In a preferred embodiment, when the pharmaceutical dosage form is multiparticulate, the individual drug-containing particles have an extension in any direction of at least 2.0 mm, more preferably at least 2.2 mm, still more preferably at least 2.5 mm, yet more preferably at least 2.8 mm, even more preferably at least 3.0 mm, most preferably at least 3.2 mm, and in particular at least 3.5 mm or 4.0 mm. Preferably, when the dosage form is multiparticulate, the individual drug-containing particles have an extension in any direction of more than 2.0 mm.

In a preferred embodiment, the pharmaceutical dosage form according to the invention is monolithic and has an extension in any direction of more than 2.0 mm; or is multiparticulate, wherein the individual drug-containing particles have an extension in any direction of more than 2.0 mm.

Particularly preferably, the pharmaceutical dosage form according to the invention is monolithic and has an extension in any direction of at least 2.0 mm; or is multiparticulate, wherein the individual drug-containing particles have an extension in any direction of at least 2.0 mm.

In another preferred embodiment,
the pharmaceutical dosage form according to the invention is monolithic and has an extension in any direction of at least 2.5 mm; or is multiparticulate, wherein the individual drug-containing particles have an extension in any direction of at least 2.2 mm; or
the pharmaceutical dosage form according to the invention is monolithic and has an extension in any direction of at least 3.0 mm; or is multiparticulate, wherein the individual drug-containing particles have an extension in any direction of at least 2.5 mm; or
the pharmaceutical dosage form according to the invention is monolithic and has an extension in any direction of at least 3.5 mm; or is multiparticulate, wherein the individual drug-containing particles have an extension in any direction of at least 2.8 mm; or
the pharmaceutical dosage form according to the invention is monolithic and has an extension in any direction of at least 4.0 mm; or is multiparticulate, wherein the individual drug-containing particles have an extension in any direction of at least 3.0 mm; or
the pharmaceutical dosage form according to the invention is monolithic and has an extension in any direction of at least 4.5 mm; or is multiparticulate, wherein the individual drug-containing particles have an extension in any direction of at least 3.2 mm; or
the pharmaceutical dosage form according to the invention is monolithic and has an extension in any direction of at least 5.0 mm; or is multiparticulate, wherein the individual drug-containing particles have an extension in any direction of at least 3.5 mm or at least 4.0 mm.

The size of particles may be determined by any conventional procedure known in the art, e.g. laser light scattering, sieve analysis, light microscopy or image analysis.

Preferably, when the pharmaceutical dosage form is multiparticulate, the plurality of particles that is contained in the pharmaceutical dosage form according to the invention has an arithmetic average weight, in the following referred to as “aaw”, wherein at least 70%, more preferably at least 75%, still more preferably at least 80%, yet more preferably at least 85%, most preferably at least 90% and in particular at least 95% of the individual particles contained in said plurality of particles has an individual weight within the range of aaw±30%, more preferably aaw±25%, still more preferably aaw±20%, yet more preferably aaw±15%, most preferably aaw±10%, and in particular aaw±5%. For example, if the pharmaceutical dosage form according to the invention contains a plurality of 100 particles and aaw of said plurality of particles is 1.00 mg, at least 75 individual particles (i.e. 75%) have an individual weight within the range of from 0.70 to 1.30 mg (1.00 mg±30%).

In a preferred embodiment, the particles, more preferably the drug-containing particles, each have a weight of less than 20 mg, more preferably less than 18 mg, still more preferably less than 16 mg, yet more preferably less than 14 mg, even more preferably less than 12 mg or less than 10 mg, most preferably less than 8 mg, and in particular less than 6 or 4 mg. According to this embodiment, all individual particles each preferably have a weight of from 1 to 19 mg, more preferably 1.5 to 15 mg, still more preferably 2.0 to 12 mg, yet more preferably 2.2 to 10 mg, even more preferably 2.5 to 8 mg, most preferably 2.8 to 6 mg and in particular 3 to 5 mg.

In another preferred embodiment, the particles, more preferably the drug-containing particles, each have a weight of 20 mg or more. According to this embodiment, all individual particles preferably each have a weight of at least 30 mg, more preferably at least 40 mg, still more preferably at least 50 mg, most preferably at least 60 mg and in particular at least 100 mg. Preferably, all individual particles each have a weight of from 20 to 1000 mg, more preferably 30 to 800 mg, still more preferably 40 to 600 mg, yet more preferably 50 to 400 mg, even more preferably 60 to 200 mg, most preferably 70 to 150 mg and in particular 80 to 120 mg. According to this embodiment, the particles of the pharmaceutical dosage form, more preferably the drug-containing particles of the pharmaceutical dosage form, preferably each have an extension in any given direction of at least 2.0 mm or 3.0 mm and have a weight of at least 20 mg.

In a preferred embodiment, when the pharmaceutical dosage form is multiparticulate, the particles are not film coated.

In another preferred embodiment, when the pharmaceutical dosage form is multiparticulate, the particles are film coated. The particles according to the invention can optionally be provided, partially or completely, with a conventional coating. The particles are preferably film coated with conventional film coating compositions. Suitable coating materials are commercially available, e.g. under the trademarks Opadry® and Eudragit®.

Examples of suitable materials include cellulose esters and cellulose ethers, such as methylcellulose (MC), hydroxypropylmethylcellulose (HPMC), hydroxypropylcellulose (HPC), hydroxyethylcellulose (HEC), sodium carboxymethylcellulose (Na-CMC), ethylcellulose (EC), cellulose acetate phthalate (CAP), hydroxypropylmethylcellulose phthalate (HPMCP); poly(meth)acrylates, such as aminoalkylmethacrylate copolymers, ethylacrylate methylmethacrylate copolymers, methacrylic acid methylmethacrylate copolymers, methacrylic acid methylmethacrylate copolymers; vinyl polymers, such as polyvinylpyrrolidone, polyvinyl-acetatephthalate, polyvinyl caprolactam, polyvinyl caprolactam-EVA copolymers, polyvinylacetate; and natural film formers.

The coating material may contain excipients such as stabilizers (e.g. surfactants such as macrogol cetostearylether, sodium dodecylsulfate, and the like). Suitable excipients of film coating materials are known to the skilled person.

In a particularly preferred embodiment, the coating is water-soluble.

Though less preferred, the coating can principally be resistant to gastric juices and dissolve as a function of the pH value of the release environment. By means of this coating, it is possible to ensure that the pharmaceutical dosage form according to the invention passes through the stomach undissolved and the active compound is only released in the intestines. The coating which is resistant to gastric juices preferably dissolves at a pH value of between 5 and 7.5. Corresponding materials and methods for the delayed release of active compounds and for the application of coatings which are resistant to gastric juices are known to the person skilled in the art, for example from “Coated Pharmaceutical dosage forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers.

A particularly preferred coating contains polyvinyl caprolactam and optionally, further excipients such as xanthan gum and/or talcum.

When the pharmaceutical dosage form is multiparticulate, the particles contain at least a pharmacologically active ingredient having psychotropic action and a polyethylene glycol graft copolymer, preferably a prolonged release matrix containing the polyethylene glycol graft copolymer as prolonged release matrix material and optionally additional prolonged release matrix material. Preferably, however, the particles further contain additional pharmaceutical excipients such as antioxidants and plasticizers.

When the pharmaceutical dosage form is multiparticulate, the particles may be e.g. loosely contained in a capsule, or the particles may be incorporated into an outer matrix material. From a macroscopic perspective, the outer matrix material preferably forms a continuous phase in which the particles are embedded as discontinuous phase.

Preferably, the outer matrix material is preferably a homogenous coherent mass, preferably a homogeneous mixture of solid constituents, in which the particles are embedded thereby spatially separating the particles from one another. While it is possible that the surfaces of particles are in contact or at least in very close proximity with one another, the plurality of particles preferably cannot be regarded as a single continuous coherent mass within the pharmaceutical dosage form.

In other words, when the pharmaceutical dosage form is multiparticulate and the particles are contained in an outer matrix material, the pharmaceutical dosage form according to the invention preferably comprises the particles as volume element(s) of a first type in which the pharmacologically active ingredient and the polyethylene glycol graft copolymer are contained, and the outer matrix material as volume element of a second type differing from the material that forms the particles, preferably containing neither pharmacologically active ingredient nor polyethylene glycol graft copolymer.

When the pharmaceutical dosage form is multiparticulate and the particles are contained in an outer matrix material, the relative weight ratio of particles to outer matrix material is not particularly limited. Preferably, said relative weight ratio is within the range of 1:1.00±0.75, more preferably 1:1.00±0.50, still more preferably 1:1.00±0.40, yet more preferably 1:1.00±0.30, most preferably 1:1.00±0.20, and in particular 1:1.00±0.10.

Preferably, the content of the outer matrix material is at least 2.5 wt.-%, at least 5 wt.-%, at least 7.5 wt.-% or at least 10 wt.-%; at least 12.5 wt.-%, at least 15 wt.-%, at least 17.5 wt.-% or at least 20 wt.-%; at least 22.5 wt.-%, at least 25 wt.-%, at least 27.5 wt.-% or at least 30 wt.-%; at least 32.5 wt.-%, at least 35 wt.-%, at least 37.5 wt.-% or at least 40 wt.-%; more preferably at least 42.5 wt.-%, at least 45 wt.-%, at least 47.5 wt.-% or at least 50 wt.-%; still more preferably at least 52.5 wt.-%, at least 55 wt.-%, at least 57.5 wt.-% or at least 60 wt.-%; yet more preferably at least 62.5 wt.-%, at least 65 wt.-%, at least 67.5 wt.-% or at least 60 wt.-%; most preferably at least 72.5 wt.-%, at least 75 wt.-%, at least 77.5 wt.-% or at least 70 wt.-%; and in particular at least 82.5 wt.-%, at least 85 wt.-%, at least 87.5 wt.-% or at least 90 wt.-%; based on the total weight of the pharmaceutical dosage form.

Preferably, the content of the outer matrix material is at most 90 wt.-%, at most 87.5 wt.-%, at most 85 wt.-%, or at most 82.5 wt.-%; more preferably at most 80 wt.-%, at most 77.5 wt.-%, at most 75 wt.-% or at most 72.5 wt.-%; still more preferably at most 70 wt.-%, at most 67.5 wt.-%, at most 65 wt.-% or at most 62.5 wt.-%; yet more preferably at most 60 wt.-%, at most 57.5 wt.-%, at most 55 wt.-% or at most 52.5 wt.-%; most preferably at most 50 wt.-%, at most 47.5 wt.-%, at most 45 wt.-% or at most 42.5 wt.-%; and in particular at most 40 wt.-%, at most 37.5 wt.-%, or at most 35 wt.-%; based on the total weight of the pharmaceutical dosage form.

Preferably, the outer matrix material is a mixture, preferably a homogeneous mixture of at least two different constituents, more preferably of at least three different constituents. In a preferred embodiment, all constituents of the outer matrix material are homogeneously distributed in the continuous phase that is formed by the outer matrix material.

Preferably, the outer matrix material is also provided in particulate form, i.e. in the course of the manufacture of the pharmaceutical dosage forms according to the invention, the constituents of the outer matrix material are preferably processed into particles, subsequently mixed with the particles that contain the pharmacologically active ingredient and the polyethylene glycol graft copolymer, and then compressed into the pharmaceutical dosage forms.

Preferably, the average size of the particles of the outer matrix material is within the range of ±60%, more preferably ±50%, still more preferably ±40%, yet more preferably ±30%, most preferably ±20%, and in particular ±10% of the average size of the particles that contain the pharmacologically active ingredient and the polyethylene glycol graft copolymer.

The particles of the outer matrix material can be manufactured by conventional methods for the preparation of aggregates and agglomerates from powder mixtures such as granulating and compacting.

In a preferred embodiment, the mixture of all constituents of the outer matrix material is blended and pre-compacted thereby yielding a pre-compacted outer matrix material.

The outer matrix material preferably does not contain any pharmacologically active ingredient.

Preferably, the outer matrix material comprises a filler or a binder. As many fillers can be regarded as binders and vice versa, for the purpose of specification “filler/binder” refers to any excipient that is suitable as filler, binder or both. Thus, the outer matrix material preferably comprises a filler/binder.

Preferred fillers (=filler/binders) are selected from the group consisting of silicium dioxide (e.g. Aerosil®), microcrystalline cellulose (e.g. Avicel®, Elcema®, Emocel®, ExCel®, Vitacell®); cellulose ether (e.g. Natrosol®, Klucel®, Methocel®, Blanose®, Pharmacoat®, Viscontran®); mannitol; dextrines; dextrose; calciumhydrogen phosphate (e.g. Emcompress®); maltodextrine (e.g. Emdex®); lactose (e.g. Fast-Flow Lactose®; Ludipress® Pharmaceutical dosage Formtose®, Zeparox®); polyvinylpyrrolidone (PVP) (e.g. Kollidone®, Polyplasdone®, Polydone®); saccharose (e.g. NuTab®, Sugar Tab®); magnesium salts (e.g. MgCO₃, MgO, MgSiO₃); starches and pretreated starches (e.g. Prejel®, Primotab® ET, Starch® 1500). Preferred binders are selected from the group consisting of alginates; chitosanes; and any of the fillers mentioned above (=fillers/binders).

Some fillers/binders may also serve other purposes. It is known, for example, that silicium dioxide exhibits excellent function as a glidant. Thus, preferably, the outer matrix material comprises a glidant such as silicium dioxide.

In a preferred embodiment, the content of the filler/binder or mixture of fillers/binders in the outer matrix material is within the range of 50±25 wt.-%, more preferably 50±20 wt.-%, still more preferably 50±15 wt.-%, yet more preferably 50±10 wt.-%, most preferably 50±7.5 wt.-%, and in particular 50±5 wt.-%, based on the total weight of outer matrix material. In another preferred embodiment, the content of the filler/binder or mixture of fillers/binders in the outer matrix material is within the range of 65±25 wt.-%, more preferably 65±20 wt.-%, still more preferably 65±15 wt.-%, yet more preferably 65±10 wt.-%, most preferably 65±7.5 wt.-%, and in particular 65±5 wt.-%, based on the total weight of outer matrix material. In still another preferred embodiment, the content of the filler/binder or mixture of fillers/binders in the outer matrix material is within the range of 80±19 wt.-%, more preferably 80±17.5 wt.-%, still more preferably 80±15 wt.-%, yet more preferably 80±10 wt.-%, most preferably 80±7.5 wt.-%, and in particular 80±5 wt.-%, based on the total weight of outer matrix material. In another preferred embodiment, the content of the filler/binder or mixture of fillers/binders in the outer matrix material is within the range of 90±9 wt.-%, more preferably 90±8 wt.-%, still more preferably 90±7 wt.-%, yet more preferably 90±6 wt.-%, most preferably 90±5 wt.-%, and in particular 90±4 wt.-%, based on the total weight of outer matrix material.

In a preferred embodiment, the content of the filler/binder or mixture of fillers/binders in the pharmaceutical dosage form is within the range of 25±24 wt.-%, more preferably 25±20 wt.-%, still more preferably 25±16 wt.-%, yet more preferably 25±12 wt.-%, most preferably 25±8 wt.-%, and in particular 25±4 wt.-%, based on the total weight of pharmaceutical dosage form. In another preferred embodiment, the content of the filler/binder or mixture of fillers/binders in the pharmaceutical dosage form is within the range of 30±29 wt.-%, more preferably 30±25 wt.-%, still more preferably 30±20 wt.-%, yet more preferably 30±15 wt.-%, most preferably 30±10 wt.-%, and in particular 30±5 wt.-%, based on the total weight of pharmaceutical dosage form. In still another preferred embodiment, the content of the filler/binder or mixture of fillers/binders in the pharmaceutical dosage form is within the range of 35±34 wt.-%, more preferably 35±28 wt.-%, still more preferably 35±22 wt.-%, yet more preferably 35±16 wt.-%, most preferably 35±10 wt.-%, and in particular 35±4 wt.-%, based on the total weight of pharmaceutical dosage form. In another preferred embodiment, the content of the filler/binder or mixture of fillers/binders in the pharmaceutical dosage form is within the range of 40±39 wt.-%, more preferably 40±32 wt.-%, still more preferably 40±25 wt.-%, yet more preferably 40±18 wt.-%, most preferably 40±11 wt.-%, and in particular 40±4 wt.-%, based on the total weight of pharmaceutical dosage form.

Preferably, the filler/binder is contained in the outer matrix material but not in the drug-containing particles of the pharmaceutical dosage form according to the invention.

Preferably, the outer matrix material comprises a diluent or lubricant, preferably selected from the group consisting of calcium stearate; magnesium stearate; glycerol monobehenate (e.g. Compritol®); Myvatex®; Precirol®; Precirol® Ato5; sodium stearylfumarate (e.g. Pruv®); and talcum. Magnesium stearate is particularly preferred. Preferably, the content of the lubricant in the outer matrix material is at most 10.0 wt.-%, more preferably at most 7.5 wt.-%, still more preferably at most 5.0 wt.-%, yet more preferably at most 2.0 wt.-%, even more preferably at most 1.0 wt.-%, and most preferably at most 0.5 wt.-%, based on the total weight of the outer matrix material and based on the total weight of pharmaceutical dosage form.

In particularly preferred embodiment, the outer matrix material comprises a combination of filler/binder and lubricant.

The outer matrix material of the pharmaceutical dosage forms according to the invention may additionally contain other excipients that are conventional in the art, e.g. diluents, binders, granulating aids, colorants, flavor additives, glidants, wet-regulating agents and disintegrants. The skilled person will readily be able to determine appropriate quantities of each of these excipients.

In a preferred embodiment, however, the outer matrix material of the pharmaceutical dosage form according to the invention consists of one or more disintegrants, one or more filler/binder's and one or more lubricants, but does not contain any other constituents.

In a particularly preferred embodiment, the outer matrix material of the pharmaceutical dosage form according to the invention does not contain one or more gel-forming agents and/or a silicone.

As used herein the term “gel-forming agent” is used to refer to a compound that, upon contact with a solvent (e.g. water), absorbs the solvent and swells, thereby forming a viscous or semi-viscous substance. Preferred gel-forming agents are not cross-linked. This substance may moderate pharmacologically active ingredient release from the embedded particles in both aqueous and aqueous alcoholic media. Upon full hydration, a thick viscous solution or dispersion is typically produced that significantly reduces and/or minimizes the amount of free solvent which can contain an amount of solubilized pharmacologically active ingredient, and which can be drawn into a syringe. The gel that is formed may also reduce the overall amount of pharmacologically active ingredient extractable with the solvent by entrapping the pharmacologically active ingredient within a gel structure. Thus the gel-forming agent may play an important role in conferring tamper-resistance to the pharmaceutical dosage forms according to the invention.

Gel-forming agents that preferably are not contained in the outer matrix material include pharmaceutically acceptable polymers, typically hydrophilic polymers, such as hydrogels. Representative examples of gel-forming agent include polyalkylene oxide such as polyethylene oxide, hydroxypropylmethyl cellulose, carbomers, poly(uronic) acids and mixtures thereof.

When the pharmaceutical dosage form comprises a prolonged release matrix in which the pharmacologically active ingredient is embedded, preferably, the overall content of the prolonged release matrix, i.e. of the prolonged release matrix material and the optionally present additional prolonged release matrix material, is within the range of from 5 to 95 wt.-%, more preferably 15 to 90 wt.-%, still more preferably 25 to 88 wt.-%, yet more preferably 35 to 86 wt.-%, even more preferably 45 to 84 wt.-%, most preferably 55 to 82 wt.-% and in particular 60 to 80 wt.-%, relative to the total weight of the pharmaceutical dosage form or, when the pharmaceutical dosage form is multiparticulate, based on the total weight of the particles that contain the pharmacologically active ingredient.

In a preferred embodiment, the content of the prolonged release matrix is at least 20 wt.-% or at least 25 wt.-%, more preferably at least 30 wt.-%, still more preferably at least 40 wt.-%, yet more preferably at least 50 wt.-% and in particular at least 60 wt.-%, either based on the total weight of the pharmaceutical dosage form or, when the pharmaceutical dosage form is multiparticulate, based on the total weight of the particles that contain the pharmacologically active ingredient.

In a preferred embodiment, the overall content of prolonged release matrix is within the range of 30±20 wt.-%, more preferably 30±15 wt.-%, most preferably 30±10 wt.-%, and in particular 30±5 wt.-%, either based on the total weight of the pharmaceutical dosage form or, when the pharmaceutical dosage form is multiparticulate, based on the total weight of the particles that contain the pharmacologically active ingredient. In another preferred embodiment, the overall content of prolonged release matrix is within the range of 35±20 wt.-%, more preferably 35±15 wt.-%, most preferably 35±10 wt.-%, and in particular 35±5 wt.-%, either based on the total weight of the pharmaceutical dosage form or, when the pharmaceutical dosage form is multiparticulate, based on the total weight of the particles that contain the pharmacologically active ingredient. In still another preferred embodiment, the overall content of prolonged release matrix is within the range of 40±20 wt.-%, more preferably 40±15 wt.-%, and most preferably 40±10 wt.-%, and in particular 40±5 wt.-%, either based on the total weight of the pharmaceutical dosage form or, when the pharmaceutical dosage form is multiparticulate, based on the total weight of the particles that contain the pharmacologically active ingredient. In yet another preferred embodiment, the overall content of prolonged release matrix is within the range of 45±20 wt.-%, more preferably 45±15 wt.-%, and most preferably 45±10 wt.-%, and in particular 45±5 wt.-%, either based on the total weight of the pharmaceutical dosage form or, when the pharmaceutical dosage form is multiparticulate, based on the total weight of the particles that contain the pharmacologically active ingredient. In even another preferred embodiment, the overall content of prolonged release matrix is within the range of 50±20 wt.-%, more preferably 50±15 wt.-%, and most preferably 50±10 wt.-%, and in particular 50±5 wt.-%, either based on the total weight of the pharmaceutical dosage form or, when the pharmaceutical dosage form is multiparticulate, based on the total weight of the particles that contain the pharmacologically active ingredient. In a further preferred embodiment, the overall content of prolonged release matrix is within the range of 55±20 wt.-%, more preferably 55±15 wt.-%, and most preferably 55±10 wt.-%, and in particular 55±5 wt.-%, either based on the total weight of the pharmaceutical dosage form or, when the pharmaceutical dosage form is multiparticulate, based on the total weight of the particles that contain the pharmacologically active ingredient. In still a further preferred embodiment, the overall content of prolonged release matrix is within the range of 60±20 wt.-%, more preferably 60±15 wt.-%, and most preferably 60±10 wt.-%, and in particular 60±5 wt.-%, either based on the total weight of the pharmaceutical dosage form or, when the pharmaceutical dosage form is multiparticulate, based on the total weight of the particles that contain the pharmacologically active ingredient. In yet a further preferred embodiment, the overall content of prolonged release matrix is within the range of 65±20 wt.-%, more preferably 65±15 wt.-%, and most preferably 65±10 wt.-%, and in particular 65±5 wt.-%, either based on the total weight of the pharmaceutical dosage form or, when the pharmaceutical dosage form is multiparticulate, based on the total weight of the particles that contain the pharmacologically active ingredient. In even a further preferred embodiment, the overall content of prolonged release matrix is within the range of 70±20 wt.-%, more preferably 70±15 wt.-%, and most preferably 70±10 wt.-%, and in particular 70±5 wt.-%, either based on the total weight of the pharmaceutical dosage form or, when the pharmaceutical dosage form is multiparticulate, based on the total weight of the particles that contain the pharmacologically active ingredient. In another further preferred embodiment, the overall content of prolonged release matrix is within the range of 75±20 wt.-%, more preferably 75±15 wt.-%, and most preferably 75±10 wt.-%, and in particular 75±5 wt.-%, either based on the total weight of the pharmaceutical dosage form or, when the pharmaceutical dosage form is multiparticulate, based on the total weight of the particles that contain the pharmacologically active ingredient. In still another preferred embodiment, the overall content of prolonged release matrix is within the range of 80±20 wt.-%, more preferably 80±15 wt.-%, and most preferably 80±10 wt.-%, and in particular 80±5 wt.-%, either based on the total weight of the pharmaceutical dosage form or, when the pharmaceutical dosage form is multiparticulate, based on the total weight of the particles that contain the pharmacologically active ingredient.

Preferably, the relative weight ratio of the prolonged release matrix to the pharmacologically active ingredient is within the range of 20:1 to 1:20, more preferably 15:1 to 1:15, still more preferably 10:1 to 1:10, yet more preferably 7:1 to 1:7, most preferably 5:1 to 1:5, and in particular 3:1 to 1:1.

Irrespective of whether the pharmaceutical dosage form is multiparticulate or not, the pharmaceutical dosage form according to the invention comprises a pharmacologically active ingredient having psychotropic action and a polyethylene glycol graft copolymer.

Particularly preferably, the pharmaceutical dosage form according to the invention comprises a prolonged release matrix containing the polyethylene glycol graft copolymer as prolonged release matrix material in which the pharmacologically active ingredient is embedded.

Preferably, the graft polymer according to the invention is a segmented copolymer with a linear backbone of one composite and randomly distributed branches of another composite. Although the side chains are preferably structurally distinct from the main chain, the individual grafted chains may be homopolymers or copolymers, alternating copolymers, random copolymers or block copolymers.

Preferably, the polyethylene glycol graft copolymer according to the invention is a bipolymer (i.e. derived from two different comonomers) or a terpolymer (i.e. derived from three different comonomers).

In a preferred embodiment, the polyethylene glycol graft copolymer comprises repetition units derived from oxirane or ethylene glycol (ethylene glycol repetition units) such that it comprises a polyethylene glycol backbone that is grafted by branches of another composite, wherein said another composite is preferably derived from vinyl acetate (vinyl acetate repetition units) and/or from vinyl caprolactam (vinyl caprolactam repetition units).

Preferably, the polyethylene glycol graft copolymer is a block copolymer wherein a polyethylene glycol backbone is grafted with block copolymers that are preferably derived from polyvinyl acetate blocks and polyvinyl caprolactam blocks. Preferably, the polyvinyl caprolactam blocks are linked to the polyethylene glycol backbone through the polyvinyl acetate blocks.

A polyvinyl caprolactam-polyvinyl acetate-polyethylene glycol graft copolymer (PCA-PVA-PEG) preferably includes a polymer comprising at least one polyvinyl caprolactam block, at least one polyvinyl acetate block, and at least one polyethylene glycol block, wherein at least one block branches from another of the type of blocks.

Suitable polyethylene glycol graft copolymers are known to the person skilled in the art and are commercially available. A particularly useful polyethylene glycol graft copolymer is Soluplus®, BASF (CAS No. 402932-23-4). Soluplus is a polyvinyl caprolactam-polyvinyl acetate-polyethylene glycol graft copolymer. It is a free flowing white to slightly yellowish granule with a faint characteristic odor and has practically no taste.

Preferably, the polyethylene glycol graft copolymer according to the invention has the general formula (I)

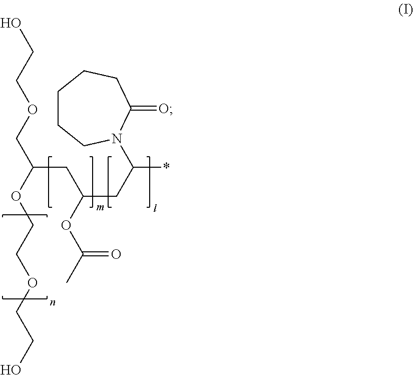

wherein

index l is 10 to 10,000, 100 to 900 100 to 500, or 500 to 900;

index m is 20 to 20,000, 150 to 1500 200 to 800, or 800 to 1500; and

index n is 30 to 30,000, 300 to 3000, 300 to 1000, 1000 to 2000, or 2000 to 3000.

Preferably, the relative molar content of the ethylene glycol repetition units within the polyethylene glycol graft copolymer is greater than the relative molar content of the vinyl acetate repetition units and/or the vinyl caprolactam repetition units within the polyethylene glycol graft copolymer.

Preferably, the polyethylene glycol graft copolymer contains at least 10 wt.-%, more preferably at least 20 wt.-%, still more preferably at least 25 wt.-%, yet more preferably at least 30 wt.-%, even more preferably at least 35 wt.-%, most preferably at least 40 wt.-% and in particular at least 45 wt.-% of ethylene glycol repetition units, relative to the total weight of the polyethylene glycol graft copolymer. Particularly preferably, the polyethylene glycol graft copolymer contains at least 50 wt.-%, more preferably at least 52 wt.-%, still more preferably at least 54 wt.-%, yet more preferably at least 55 wt.-%, even more preferably at least 56 wt.-%, most preferably at least 57 wt.-% and in particular at least 58 wt.-% of ethylene glycol repetition units, relative to the total weight of the polyethylene glycol graft copolymer. In another preferred embodiment, the polyethylene glycol graft copolymer contains at least 60 wt.-%, more preferably at least 62 wt.-%, still more preferably at least 64 wt.-%, yet more preferably at least 66 wt.-%, even more preferably at least 68 wt.-%, most preferably at least 69 wt.-% and in particular at least 70 wt.-% of ethylene glycol repetition units, relative to the total weight of the polyethylene glycol graft copolymer. In still another preferred embodiment, the polyethylene glycol graft copolymer contains at least 72 wt.-%, more preferably at least 75 wt.-%, still more preferably at least 78 wt.-%, yet more preferably at least 80 wt.-%, even more preferably at least 82 wt.-%, most preferably at least 84 wt.-% and in particular at least 86 wt.-% of ethylene glycol repetition units, relative to the total weight of the polyethylene glycol graft copolymer.

Preferably, the polyethylene glycol graft copolymer contains from 30 to 99 wt.-% of ethylene glycol repetition units, relative to the total weight of the polyethylene glycol graft copolymer. Particularly preferably, the polyethylene glycol graft copolymer contains from 50 to 95 wt.-% of ethylene glycol repetition units, relative to the total weight of the polyethylene glycol graft copolymer.

In a preferred embodiment, the polyethylene glycol graft copolymer contains 30±25 wt.-%, more preferably 30±20 wt.-%, still more preferably 30±17 wt.-%, yet more preferably 30±13 wt.-%, even more preferably 30±10 wt.-%, most preferably 30±7 wt.-% and in particular 30±5 wt.-% of ethylene glycol repetition units, relative to the total weight of the polyethylene glycol graft copolymer. In another preferred embodiment, the polyethylene glycol graft copolymer contains 40±35 wt.-%, more preferably 40±30 wt.-%, still more preferably 40±25 wt.-%, yet more preferably 40±20 wt.-%, even more preferably 40±15 wt.-%, most preferably 40±10 wt.-% and in particular 40±5 wt.-% of ethylene glycol repetition units, relative to the total weight of the polyethylene glycol graft copolymer. In still another preferred embodiment, the polyethylene glycol graft copolymer contains 50±45 wt.-%, more preferably 50±35 wt.-%, still more preferably 50±25 wt.-%, yet more preferably 50±20 wt.-%, even more preferably 50±15 wt.-%, most preferably 50±10 wt.-% and in particular 50±5 wt.-% of ethylene glycol repetition units, relative to the total weight of the polyethylene glycol graft copolymer. In yet another preferred embodiment, the polyethylene glycol graft copolymer contains 60±35 wt.-%, more preferably 60±30 wt.-%, still more preferably 60±25 wt.-%, yet more preferably 60±20 wt.-%, even more preferably 60±15 wt.-%, most preferably 60±10 wt.-% and in particular 60±5 wt.-% of ethylene glycol repetition units, relative to the total weight of the polyethylene glycol graft copolymer. In a further preferred embodiment, the polyethylene glycol graft copolymer contains 70±25 wt.-%, more preferably 70±20 wt.-%, still more preferably 70±17 wt.-%, yet more preferably 70±13 wt.-%, even more preferably 70±10 wt.-%, most preferably 70±7 wt.-% and in particular 70±5 wt.-% of ethylene glycol repetition units, relative to the total weight of the polyethylene glycol graft copolymer. In still a further preferred embodiment, the polyethylene glycol graft copolymer contains 80±15 wt.-%, more preferably 80±12 wt.-%, still more preferably 80±10 wt.-%, yet more preferably 80±8 wt.-%, even more preferably 80±6 wt.-%, most preferably 80±4 wt.-% and in particular 80±2 wt.-% of ethylene glycol repetition units, relative to the total weight of the polyethylene glycol graft copolymer. In yet a further preferred embodiment, the polyethylene glycol graft copolymer contains 90±15 wt.-%, more preferably 90±12 wt.-%, still more preferably 90±10 wt.-%, yet more preferably 90±8 wt.-%, even more preferably 90±6 wt.-%, most preferably 90±4 wt.-% and in particular 90±2 wt.-% of ethylene glycol repetition units, relative to the total weight of the polyethylene glycol graft copolymer.

Preferably, the molar ratio of the vinyl acetate repetition units to the vinyl caprolactam repetition units is within the range of from 1000:1 to 1:1000, more preferably from 900:1 to 1:900, still more preferably from 500:1 to 1:500, yet more preferably from 300:1 to 1:300, even more preferably from 200:1 to 1:200, most preferably from 100:1 to 100:1, and in particular 10:1 to 1:10.

Preferably, the polyethylene glycol graft copolymer has an average molecular weight, as determined by gel permeation chromatography, within the range of 115,000±100,000 g/mol, more preferably 115,000±80,000 g/mol, still more preferably 115,000±70,000 g/mol, yet more preferably 115,000±60,000 g/mol, even more preferably 115,000±50,000 g/mol, most preferably 115,000±40,000 g/mol, and in particular 115,000±35,000 g/mol. In some embodiments, the polyethylene glycol graft copolymer has an average molecular weight of 1,000 g/mol to 5,000,000 g/mol, 10,000 g/mol to 500,000 g/mol, or 90,000 g/mol to 140,000 g/mmol.

Preferably, the polyethylene glycol graft copolymer has a critical micelle concentration within the range of 8±6 ppm, more preferably 8±5 ppm, still more preferably 8±4 ppm, yet more preferably 8±3 ppm, even more preferably 8±2 ppm, most preferably 8±1.5 ppm, and in particular 8±1 ppm.

Preferably, the polyethylene glycol graft copolymer has a glass transition temperature within the range of 70±35° C., more preferably 70±30° C., still more preferably 70±25° C., yet more preferably 70±20° C., even more preferably 70±15° C., most preferably 70±10° C., and in particular 70±5° C.

The polyethylene glycol graft copolymer may comprise a single polyethylene glycol graft copolymer, or a mixture (blend) of different polyethylene glycol graft copolymers, such as two, three, four or five polyethylene glycol graft copolymers, e.g., polyethylene glycol graft copolymers of the same chemical nature but different molecular weights, polyethylene glycol graft copolymers of different chemical nature but same molecular weights, or polyethylene glycol graft copolymers of different chemical nature as well as different molecular weights. In a preferred embodiment, the polyethylene glycol graft copolymer comprises a single polyethylene glycol graft copolymer.

In a preferred embodiment, the polyethylene glycol graft copolymer is homogeneously distributed in the pharmaceutical dosage form according to the invention.

When the pharmaceutical dosage form is multiparticulate, the polyethylene glycol graft copolymer is preferably homogeneously distributed in the particles according to the invention that contain the pharmacologically active ingredient. Preferably, the pharmacologically active ingredient and the polyethylene glycol graft copolymer are intimately homogeneously distributed in the pharmaceutical dosage form and the particles, respectively, so that the pharmaceutical dosage form and the particles, respectively, do not contain any segments where either pharmacologically active ingredient is present in the absence of polyethylene glycol graft copolymer or where polyethylene glycol graft copolymer is present in the absence of pharmacologically active ingredient.

When the pharmaceutical dosage form and the particles, respectively, are film coated, the polyethylene glycol graft copolymer is preferably homogeneously distributed in the core of the pharmaceutical dosage form and the particles, respectively, i.e. the film coating preferably does not contain polyethylene glycol graft copolymer. Nonetheless, the film coating as such may of course contain one or more polymers, which however, preferably differ from the polyethylene glycol graft copolymer contained in the core.

The content of the polyethylene glycol graft copolymer is preferably within the range of from 25 to 95 wt.-%, more preferably 25 to 94 wt.-%, still more preferably 25 to 93 wt.-%, yet more preferably 25 to 92 wt.-%, most preferably 25 to 91 wt.-%, and in particular 25 to 90 wt.-%, relative to the total weight of the pharmaceutical dosage form. When the pharmaceutical dosage form is multiparticulate, these percent values preferably are related to the total weight of the particles, not to the total weight of the pharmaceutical dosage form.

In a particularly preferred embodiment, the content of the polyethylene glycol graft copolymer is within the range of from 25 to 80 wt.-%, more preferably 25 to 78 wt.-%, still more preferably 30 to 76 wt.-%, yet more preferably 35 to 74 wt.-%, most preferably 40 to 72 wt.-% and in particular 45 to 70 wt.-%, relative to the total weight of the pharmaceutical dosage form or, when the pharmaceutical dosage form is multiparticulate, relative to the total weight of the particles that contain the pharmacologically active ingredient.

In a preferred embodiment, the content of the polyethylene glycol graft copolymer is at least 30 wt.-%, more preferably at least 35 wt.-%, still more preferably at least 40 wt.-%, yet more preferably at least 45 wt.-%, even more preferably at least 50, most preferably at least 55 wt.-% and in particular at least 60 wt.-%, relative to the total weight of the pharmaceutical dosage form or, when the pharmaceutical dosage form is multiparticulate, relative to the total weight of the particles that contain the pharmacologically active ingredient.

In a preferred embodiment, the content of the polyethylene glycol graft copolymer is 30±5 wt.-%, relative to the total weight of the pharmaceutical dosage form or, when the pharmaceutical dosage form is multiparticulate, relative to the total weight of the particles that contain the pharmacologically active ingredient. In another preferred embodiment, the content of the polyethylene glycol graft copolymer is 40±15 wt.-%, preferably 40±10 wt.-% and in particular 40±5 wt.-%, relative to the total weight of the pharmaceutical dosage form or, when the pharmaceutical dosage form is multiparticulate, relative to the total weight of the particles that contain the pharmacologically active ingredient. In yet another preferred embodiment, the content of the polyethylene glycol graft copolymer is 50±25 wt.-%, yet more preferably 50±20 wt.-%, even more preferably 50±15 wt.-%, most preferably 50±10 wt.-% and in particular 50±5 wt.-%, relative to the total weight of the pharmaceutical dosage form or, when the pharmaceutical dosage form is multiparticulate, relative to the total weight of the particles that contain the pharmacologically active ingredient. In even another preferred embodiment, the content of the polyethylene glycol graft copolymer is 55±20 wt.-%, even more preferably 55±15 wt.-%, most preferably 55±10 wt.-% and in particular 55±5 wt.-%, relative to the total weight of the pharmaceutical dosage form or, when the pharmaceutical dosage form is multiparticulate, relative to the total weight of the particles that contain the pharmacologically active ingredient. In a further preferred embodiment, the content of the polyethylene glycol graft copolymer is 60±35 wt.-%, more preferably 60±30 wt.-%, still more preferably 60±25 wt.-%, yet more preferably 60±20 wt.-%, even more preferably 60±15 wt.-%, most preferably 60±10 wt.-% and in particular 60±5 wt.-%, relative to the total weight of the pharmaceutical dosage form or, when the pharmaceutical dosage form is multiparticulate, relative to the total weight of the particles that contain the pharmacologically active ingredient. In still a further preferred embodiment, the content of the polyethylene glycol graft copolymer is 65±30 wt.-%, more preferably 65±25 wt.-%, still more preferably 65±20 wt.-%, yet more preferably 65±15 wt.-%, even more preferably 65±10 wt.-%, most preferably 65±7 wt.-% and in particular 65±5 wt.-%, relative to the total weight of the pharmaceutical dosage form or, when the pharmaceutical dosage form is multiparticulate, relative to the total weight of the particles that contain the pharmacologically active ingredient. In yet a further preferred embodiment, the content of the polyethylene glycol graft copolymer is 70±25 wt.-%, more preferably 70±20 wt.-%, still more preferably 70±17 wt.-%, yet more preferably 70±13 wt.-%, even more preferably 70±10 wt.-%, most preferably 70±7 wt.-% and in particular 70±5 wt.-%, relative to the total weight of the pharmaceutical dosage form or, when the pharmaceutical dosage form is multiparticulate, relative to the total weight of the particles that contain the pharmacologically active ingredient. In even a further preferred embodiment, the content of the polyethylene glycol graft copolymer is 80±15 wt.-%, more preferably 80±12 wt.-%, still more preferably 80±10 wt.-%, yet more preferably 80±8 wt.-%, even more preferably 80±6 wt.-%, most preferably 80±4 wt.-% and in particular 80±2 wt.-%, relative to the total weight of the pharmaceutical dosage form or, when the pharmaceutical dosage form is multiparticulate, relative to the total weight of the particles that contain the pharmacologically active ingredient.

When the pharmaceutical dosage form comprises a prolonged release matrix containing the polyethylene glycol graft copolymer as prolonged release matrix material, the content of the polyethylene glycol graft copolymer is preferably within the range of from 5 to 100 wt.-%, more preferably 20 to 98 wt.-%, still more preferably 35 to 96 wt.-%, yet more preferably 45 to 95 wt.-%, even more preferably 55 to 94 wt.-%, most preferably 65 to 93 wt.-%, and in particular 75 to 92 wt.-%, relative to the total weight of the prolonged release matrix, i.e. total weight of the prolonged release matrix material and the optionally present additional prolonged release matrix material.

Preferably, the relative weight ratio of the polyethylene glycol graft copolymer to the pharmacologically active ingredient is within the range of 20:1 to 1:20, more preferably 15:1 to 1:15, still more preferably 10:1 to 1:10, yet more preferably 7:1 to 1:7, most preferably 5:1 to 1:5, and in particular 3:1 to 1:1.

In a preferred embodiment, when the pharmaceutical dosage form according to the invention comprises a prolonged release matrix, the prolonged release matrix in turn comprises an additional prolonged release matrix material besides the prolonged release matrix material, i.e. the polyethylene glycol graft copolymer. Thus, the additional prolonged release matrix material is to be distinguished from the prolonged release matrix material of the prolonged release matrix of the pharmaceutical dosage form according to the invention.

Preferably, the additional prolonged release matrix material is a polymer selected from the group comprising polyalkylene oxides, acrylic polymers, crosslinked acrylic polymers, mixtures of polyvinyl pyrrolidone and polyvinyl acetate, waxy materials, polyalkylene glycols and natural polysaccharides, such as celluloses, cellulose derivatives and xanthan gum.

The content of the additional prolonged release matrix material is preferably within the range of from 1 to 90 wt.-%, more preferably 2 to 80 wt.-%, still more preferably 3 to 70 wt.-%, yet more preferably 3.5 to 60 wt.-%, even more preferably 4 to 50 wt.-%, most preferably 4.5 to 40 wt.-%, and in particular 5 to 30 wt.-%, relative to the total weight of the prolonged release matrix.

In a preferred embodiment, the content of the additional prolonged release matrix material is at least 2 wt.-%, more preferably at least 5 wt.-%, still more preferably at least 10 wt.-%, yet more preferably at least 15 wt.-% and in particular at least 20 wt.-%, either based on the total weight of the pharmaceutical dosage form or, when the pharmaceutical dosage form is multiparticulate, based on the total weight of the particles that contain the pharmacologically active ingredient.

The overall content of additional prolonged release matrix material is preferably within the range of from 1 to 60 wt.-%, more preferably 2 to 45 wt.-%, still more preferably 3 to 35 wt.-%, yet more preferably 4 to 28 wt.-%, even more preferably 5 to 25 wt.-%, most preferably 5 to 22 wt.-%, and in particular 5 to 20 wt.-%, either based on the total weight of the pharmaceutical dosage form or, when the pharmaceutical dosage form is multiparticulate, based on the total weight of the particles that contain the pharmacologically active ingredient.

In a preferred embodiment, the overall content of additional prolonged release matrix material is within the range of 5±4 wt.-%, more preferably 5±3 wt.-%, most preferably 5±2 wt.-%, and in particular 5±1 wt.-%, either based on the total weight of the pharmaceutical dosage form or, when the pharmaceutical dosage form is multiparticulate, based on the total weight of the particles that contain the pharmacologically active ingredient. In another preferred embodiment, the overall content of additional prolonged release matrix material is within the range of 7.5±6 wt.-%, more preferably 7.5±4 wt.-%, most preferably 7.5±3 wt.-%, and in particular 7.5±2 wt.-%, either based on the total weight of the pharmaceutical dosage form or, when the pharmaceutical dosage form is multiparticulate, based on the total weight of the particles that contain the pharmacologically active ingredient. In still another preferred embodiment, the overall content of additional prolonged release matrix material is within the range of 10±8 wt.-%, more preferably 10±6 wt.-%, most preferably 10±4 wt.-%, and in particular 10±2 wt.-%, either based on the total weight of the pharmaceutical dosage form or, when the pharmaceutical dosage form is multiparticulate, based on the total weight of the particles that contain the pharmacologically active ingredient. In yet another preferred embodiment, the overall content of additional prolonged release matrix material is within the range of 15±12 wt.-%, more preferably 15±10 wt.-%, most preferably 15±7 wt.-%, and in particular 15±3 wt.-%, either based on the total weight of the pharmaceutical dosage form or, when the pharmaceutical dosage form is multiparticulate, based on the total weight of the particles that contain the pharmacologically active ingredient. In even another preferred embodiment, the overall content of additional prolonged release matrix material is within the range of 20±16 wt.-%, more preferably 20±12 wt.-%, most preferably 20±8 wt.-%, and in particular 20±4 wt.-%, either based on the total weight of the pharmaceutical dosage form or, when the pharmaceutical dosage form is multiparticulate, based on the total weight of the particles that contain the pharmacologically active ingredient. In a further preferred embodiment, the overall content of additional prolonged release matrix material is within the range of 25±20 wt.-%, more preferably 25±15 wt.-%, most preferably 25±10 wt.-%, and in particular 25±5 wt.-%, either based on the total weight of the pharmaceutical dosage form or, when the pharmaceutical dosage form is multiparticulate, based on the total weight of the particles that contain the pharmacologically active ingredient. In still a further preferred embodiment, the overall content of additional prolonged release matrix material is within the range of 30±20 wt.-%, more preferably 30±15 wt.-%, most preferably 30±10 wt.-%, and in particular 30±5 wt.-%, either based on the total weight of the pharmaceutical dosage form or, when the pharmaceutical dosage form is multiparticulate, based on the total weight of the particles that contain the pharmacologically active ingredient. In yet a further preferred embodiment, the overall content of additional prolonged release matrix material is within the range of 35±20 wt.-%, more preferably 35±15 wt.-%, most preferably 35±10 wt.-%, and in particular 35±5 wt.-%, either based on the total weight of the pharmaceutical dosage form or, when the pharmaceutical dosage form is multiparticulate, based on the total weight of the particles that contain the pharmacologically active ingredient. In even a further preferred embodiment, the overall content of additional prolonged release matrix material is within the range of 40±20 wt.-%, more preferably 40±15 wt.-%, and most preferably 40±10 wt.-%, and in particular 40±5 wt.-%, either based on the total weight of the pharmaceutical dosage form or, when the pharmaceutical dosage form is multiparticulate, based on the total weight of the particles that contain the pharmacologically active ingredient. In another preferred embodiment, the overall content of additional prolonged release matrix material is within the range of 45±20 wt.-%, more preferably 45±15 wt.-%, and most preferably 45±10 wt.-%, and in particular 45±5 wt.-%, either based on the total weight of the pharmaceutical dosage form or, when the pharmaceutical dosage form is multiparticulate, based on the total weight of the particles that contain the pharmacologically active ingredient. In still another preferred embodiment, the overall content of additional prolonged release matrix material is within the range of 50±20 wt.-%, more preferably 50±15 wt.-%, and most preferably 50±10 wt.-%, and in particular 50±5 wt.-%, either based on the total weight of the pharmaceutical dosage form or, when the pharmaceutical dosage form is multiparticulate, based on the total weight of the particles that contain the pharmacologically active ingredient.

Preferably, the relative weight ratio of the additional prolonged release matrix material to the pharmacologically active ingredient is within the range of 20:1 to 1:20, more preferably 10:1 to 1:15, still more preferably 7:1 to 1:10, yet more preferably 5:1 to 1:7, most preferably 1:1 to 1:5, and in particular 1:2 to 1:5.

Preferably, the relative weight ratio of the additional prolonged release matrix material to the prolonged release matrix material of the prolonged release matrix is within the range of 20:1 to 1:20, more preferably 10:1 to 1:18, still more preferably 7:1 to 1:16, yet more preferably 5:1 to 1:14, most preferably 1:1 to 1:12, and in particular 1:5 to 1:10.

In a preferred embodiment, the pharmaceutical dosage form according to the invention comprises a prolonged release matrix which in turn comprises
(i) a polyethylene glycol graft copolymer; and
(ii) an additional prolonged release matrix material which is preferably a polymer selected from the group consisting of cellulose ethers, polyalkylene oxides, crosslinked acrylic polymers and matrices based on polyvinyl acetate and polyvinyl pyrrolidone;
   wherein
(iii) the relative weight content of the prolonged release matrix material is preferably greater than the relative weight content of the additional prolonged release matrix material.

In a preferred embodiment, the additional prolonged release matrix material is a polyalkylene oxide, preferably a polyethylene oxide, particularly preferably having a weight average molecular weight of at least 500,000 g/mol.

When the additional prolonged release matrix material of the prolonged release matrix comprises a polyalkylene oxide, it preferably does not additionally comprise any other additional prolonged release matrix material.

Preferably, the polyalkylene oxide is selected from polymethylene oxide, polyethylene oxide and polypropylene oxide, or copolymers or mixtures thereof.

Preferably, the polyalkylene oxide has a weight average molecular weight (M_{W}), preferably also a viscosity average molecular weight (M_{η}) of more than 200,000 g/mol or at least 500,000 g/mol, preferably at least 1,000,000 g/mol or at least 2,500,000 g/mol, more preferably in the range of 1,000,000 g/mol to 15,000,000 g/mol, and most preferably in the range of 5,000,000 g/mol to 10,000,000 g/mol. Suitable methods to determine M_{W} and M_{η} are known to a person skilled in the art. M_{η} is preferably determined by rheological measurements, whereas M_{W} can be determined by gel permeation chromatography (GPC).

Preferably, the molecular weight dispersity M_{W}/M_{η} of the polyalkylene oxide is within the range of 2.5±2.0, more preferably 2.5±1.5, still more preferably 2.5±1.0, yet more preferably 2.5±0.8, most preferably 2.5±0.6, and in particular 2.5±0.4.

The polyalkylene oxide preferably has a viscosity at 25° C. of 30 to 17,600 mPa·s, more preferably 55 to 17,600 mPa·s, still more preferably 600 to 17,600 mPa·s, yet more preferably 4,500 to 17,600 mPa·s, even more preferably 4,500 to 12,000 mPa·s, most preferably 5,000 to 10,500 mPa·s and in particular 5,500 to 7,500 mPa·s or 7,500 to 10,000 mPa·s, measured in a 1 wt.-% aqueous solution.

The polyalkylene oxide may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

For the purpose of specification, a polyalkylene glycol has a molecular weight of up to 20,000 g/mol whereas a polyalkylene oxide has a molecular weight of more than 20,000 g/mol. Preferably, the weight average over all molecular weight of all polyalkylene oxides that are contained in the pharmaceutical dosage form is more than 200,000 g/mol. Thus, polyalkylene glycols, if any, are preferably not taken into consideration when determining the weight average molecular weight of polyalkylene oxide.

In a particularly preferred embodiment, the additional prolonged release matrix material is a polyalkylene oxide, more preferably a polyethylene oxide, having a weight average molecular weight (M_{W}), preferably also a viscosity average molecular weight (M_{η}) in the range of 5,000,000 g/mol to 10,000,000 g/mol.

The overall content of polyalkylene oxide is preferably within the range of from 1 to 60 wt.-%, more preferably 3 to 45 wt.-%, still more preferably 5 to 35 wt.-%, yet more preferably 7 to 28 wt.-%, even more preferably 8 to 25 wt.-%, most preferably 9 to 22 wt.-%, and in particular 10 to 20 wt.-%, either based on the total weight of the pharmaceutical dosage form or, when the pharmaceutical dosage form is multiparticulate, based on the total weight of the particles that contain the pharmacologically active ingredient.

In a preferred embodiment, the overall content of polyalkylene oxide is within the range of 15±12 wt.-%, more preferably 15±10 wt.-%, most preferably 15±7 wt.-%, and in particular 15±3 wt.-%, either based on the total weight of the pharmaceutical dosage form or, when the pharmaceutical dosage form is multiparticulate, based on the total weight of the particles that contain the pharmacologically active ingredient.

In a preferred embodiment, the additional prolonged release matrix material is a mixture of polyvinyl pyrrolidone and polyvinyl acetate.

When the additional prolonged release matrix material of the prolonged release matrix comprises a mixture of polyvinyl pyrrolidone and polyvinyl acetate, it preferably does not additionally comprise any other additional prolonged release matrix material.

Preferably, the mixture of polyvinyl pyrrolidone and polyvinyl acetate contains 10 to 30 wt.-% polyvinyl pyrrolidone and 70 to 90 wt.-% of polyvinyl acetate, more preferably 18 to 21 wt.-% polyvinyl pyrrolidone and 75 to 85 wt.-% of polyvinyl acetate and most preferably 19 wt.-% polyvinyl pyrrolidone and 80 wt.-% of polyvinyl acetate.

The weight ratio between polyvinyl acetate and polyvinyl pyrrolidone preferably is in the range from 20:1 to 1:20, more preferably 16:1 to 1:10, still more preferably 13:1 to 1:5, yet more preferably 10:1 to 1:2, even more preferably 7:1 to 1:1, most preferably 5:1 to 2:1 and in particular 4.5:1 to 3.5:1.

Preferably, the polyvinyl acetate has a weight average molecular weight (M_{W}) of 450,000±100,000 g/mol, more preferably 450,000±80,000 g/mol, still more preferably 450,000±50,000 g/mol, yet more preferably 450,000±10,000 g/mol, even more preferably 450,000±1,000 g/mol, most preferably 450,000±500 g/mol and in particular 450,000±100 g/mol. M_{W} can be determined by gel permeation chromatography (GPC).

Preferably, the polyvinyl pyrrolidone has a weight average molecular weight (M_{W}) of 50,000±10,000 g/mol, more preferably 50,000±8,000 g/mol, still more preferably 50,000±5,000 g/mol, yet more preferably 50,000±1,000 g/mol, even more preferably 50,000±800 g/mol, most preferably 50,000±500 g/mol and in particular 50,000±100 g/mol.

The weight average molecular weight (M_{W}) of the mixture of polyvinyl pyrrolidone and polyvinyl acetate can be expressed as K-value according to the method described in the USP and Ph. Eur. monographs “Povidone”, measured in a 1% solution in tetrahydrofurane, wherein the K-value preferably is in the range of from 40 to 80, more preferably 45 to 78, still more preferably 50 to 75, most preferably 55 to 70 and in particular 60 to 65.

Preferably, the glass transition temperature (T_{g}) of the mixture of polyvinyl pyrrolidone and polyvinyl acetate is in the range of 35±10° C., more preferably 35±6° C. and most preferably 35±3° C.

In a particularly preferred embodiment, the additional prolonged release matrix material is a mixture of polyvinyl pyrrolidone and polyvinyl acetate, wherein said mixture has a K-value in the range of from 60 to 65, measured in a 1% solution in tetrahydrofurane according to the method described in the USP and Ph. Eur. monographs “Povidone” and/or wherein the weight ratio between polyvinyl acetate and polyvinyl pyrrolidone is in the range of 4.5:1 to 3.5:1.

The overall content of the mixture of polyvinyl pyrrolidone and polyvinyl acetate is preferably within the range of from 1.0 to 60 wt.-%, more preferably 2.0 to 50 wt.-%, still more preferably 3.0 to 40 wt.-%, yet more preferably 3.5 to 30 wt.-%, even more preferably 4.0 to 25 wt.-%, most preferably 4.5 to 20 wt.-%, and in particular 5 to 15 wt.-%, either based on the total weight of the pharmaceutical dosage form or, when the pharmaceutical dosage form is multiparticulate, based on the total weight of the particles that contain the pharmacologically active ingredient.

In a preferred embodiment, the overall content of the mixture of polyvinyl pyrrolidone and polyvinyl acetate is within the range of 10±8 wt.-%, more preferably 10±6 wt.-%, most preferably 10±4 wt.-%, and in particular 10±2 wt.-%, either based on the total weight of the pharmaceutical dosage form or, when the pharmaceutical dosage form is multiparticulate, based on the total weight of the particles that contain the pharmacologically active ingredient.

Mixtures of polyvinyl pyrrolidone and polyvinyl acetate that are suitable for use in the pharmaceutical dosage forms according to the invention are commercially available, e.g. from BASF, such as Kollidon® SR. For details concerning the properties of this product, it can be referred to e.g. the product specification.

In another preferred embodiment, the additional prolonged release matrix material is an acrylic polymer.

When the additional prolonged release matrix material of the prolonged release matrix comprises an acrylic polymer, it preferably does not additionally comprise any other additional prolonged release matrix material.

Preferably, the acrylic polymer has a weight average molecular weight within the range of from 100,000 g/mol to 2,000,000 g/mol. In a preferred embodiment, the acrylic polymer has a weight average molecular weight (M_{W}) or viscosity average molecular weight (M_{η}) of at least 150,000 or at least 200,000 g/mol, preferably at least 250,000 g/mol or at least 300,000 g/mol, more preferably in the range of 300,000 g/mol to 2,000,000 g/mol, and most preferably in the range of 300,000 g/mol to 1,000,000 g/mol. Suitable methods to determine M_{W} and M_{η} are known to a person skilled in the art. M_{η} is preferably determined by rheological measurements, whereas M_{W} can be determined by gel permeation chromatography (GPC).

The acrylic polymer can be a nonionic acrylic polymer or an ionic acrylic polymer. For the purpose of specification, “nonionic polymer” refers to a polymer not containing more than 1 mole.-% ionic, i.e. anionic or cationic, monomer units, preferably containing no ionic monomer units at all.

In a preferred embodiment, the additional prolonged release matrix material is an ionic acrylic polymer.

Preferred ionic acrylic polymers are anionic acrylic polymers. Preferred anionic acrylic polymers include but are not limited to homopolymers or copolymers of one or two different C₁₋₄-alkyl (meth)acrylate monomers and copolymerizable anionic monomers such as acrylic acid.

For the purpose of the specification, “(meth)acryl” refers to acryl as well as methacryl.

In a preferred embodiment, the additional prolonged release matrix material is an anionic acrylic polymer, preferably polyacrylic acid. According to this embodiment, the polyacrylic acid preferably has a viscosity within the range of 2,000 to 20,000 mPa·s, more preferably 3,000 to 18,000 mPa·s, still more preferably 3,500 to 16,000 mPa·s, yet more preferably 3,600 to 14,000 mPa·s, even more preferably 3,700 to 13,000 mPa·s, most preferably 3,800 to 12,000, and in particular 4,000 to 11,000 mPa·s, measured with a Brookfield RVT, 20 rpm, spindle no. 5 at 25° C. and 0.5 wt.-% neutralized to pH 7.3-7.8.

The acrylic polymer, preferably the anionic acrylic polymer, more preferably the polyacrylic acid polymer can optionally be crosslinked. Preferred crosslinking agents include allyl pentaerythritol, allyl sucrose, ethylene glycol di(methacrylate), methylenebisacrylamide and divinyl benzene.

In a particularly preferred embodiment, the anionic acrylic polymer is a polyacrylic acid polymer which is crosslinked, preferably with ally pentaerythritol, and has a viscosity of 4,000 to 11,000 mPa·s, measured with a Brookfield RVT, 20 rpm, spindle no. 5 at 25° C. and 0.5 wt.-% neutralized to pH 7.3-7.8.

Preferably, the overall content of anionic acrylic polymer, preferably polyacrylic acid, more preferably crosslinked polyacrylic acid, is within the range of from 1.0 to 60 wt.-%, more preferably 2.0 to 50 wt.-%, still more preferably 3.0 to 40 wt.-%, yet more preferably 3.5 to 30 wt.-%, even more preferably 4.0 to 20 wt.-%, most preferably 4.5 to 15 wt.-%, and in particular 5.0 to 12 wt.-%, either based on the total weight of the pharmaceutical dosage form or, when the pharmaceutical dosage form is multiparticulate, based on the total weight of the particles that contain the pharmacologically active ingredient.

Polyacrylic acid polymers that are suitable for use in the pharmaceutical dosage forms according to the invention are commercially available, e.g. from Lubrizol, such as Carbopol® 71G, Carbopol® 971P, Carbopol® 981 and Carbopol® 941. For details concerning the properties of these products, it can be referred to e.g. the product specification.

Other preferred anionic acrylic polymers are ternary copolymers of methyl acrylate, methyl methacrylate and methacrylic acid. Preferably, the anionic acrylic polymer has a weight average molecular weight within the range of 280,000±250,000 g/mol, more preferably 280,000±200,000 g/mol, still more preferably 280,000±180,000 g/mol, yet more preferably 280,000±160,000 g/mol, even more preferably 280,000±140,000 g/mol, most preferably 280,000±120,000 g/mol, and in particular 280,000±100,000 g/mol.

Further preferred ionic acrylic polymers are cationic acrylic polymers. Preferred cationic acrylic polymers include but are not limited to copolymers of one or two different C₁₋₄-alkyl (meth)acrylate monomers and copolymerizable cationic monomers such as trimethylammonioethyl methacrylate chloride. Preferred representatives are ternary copolymers of ethyl acrylate, methyl methacrylate and a low content of methacrylic acid ester with quaternary ammonium groups, preferably trimethylammonioethyl methacrylate chloride. Preferably, the cationic acrylic polymer has a weight average molecular weight within the range of 32,000±30,000 g/mol, more preferably 32,000±27,000 g/mol, still more preferably 32,000±23,000 g/mol, yet more preferably 32,000±20,000 g/mol, even more preferably 32,000±17,000 g/mol, most preferably 32,000±13,000 g/mol, and in particular 32,000±10,000 g/mol.

In another preferred embodiment, the additional prolonged release matrix material is a nonionic acrylic polymer.

Nonionic acrylic polymers that are suitable for use in the pharmaceutical dosage forms according to the invention are commercially available, e.g. from Evonik. For example, Eudragit® NE30D, Eudragit® NE40D and Eudragit® NM30D, which are provided as aqueous dispersions of poly(ethyl acrylate-co-methyl methacrylate) 2:1, may be used in the pharmaceutical dosage form according to the invention. For details concerning the properties of these products, it can be referred to e.g. the product specification.

In another preferred embodiment, the additional prolonged release matrix material is a waxy material.

Preferably, the waxy material is selected from the group consisting of
glycerides, especially monoglycerides, diglycerides, triglycerides,
esters of fatty acids with fatty alcohols, and
paraffins.

When the additional prolonged release matrix material of the prolonged release matrix comprises a waxy material, it preferably does not additionally comprise any other additional prolonged release matrix material.

As used herein a “waxy material” refers to a material which melts into liquid form having low viscosity upon heating and sets again to a solid state upon cooling. Preferably, the waxy material has a melting point of at least 30° C., more preferably at least 35° C., still more preferably at least 40° C., yet more preferably at least 45° C., even more preferably at least 50° C., most preferably at least 55° C., and in particular at least 60° C.

When the waxy material is or comprises a monoglyceride, diglyceride, triglyceride or a mixture thereof, it is preferably a mono-, di- or triester of glycerol and carboxylic acids, whereas the carboxylic acid is preferably selected from the group consisting of fatty acids, hydroxy fatty acids and aromatic acids.

Preferred glycerides of fatty acids include monoglycerides, diglycerides, triglycerides, and mixtures thereof; preferably of C₆ to C₂₂ fatty acids. Especially preferred are partial glycerides of the C₁₆ to C₂₂ fatty acids such as glycerol behenat, glycerol palmitostearate, glycerol monostearate, glycerol trimyristate and glycerol distearate.

The term “fatty acid” is well acknowledged in the art and includes for example unsaturated representatives such as myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, and docosahexaenoic acid; as well as saturated representatives such as caprylic acid, capric acid, lauric acid, myristic acid, palmitic acid, stearic acid, arachidic acid, behenic acid, lignoceric acid, and cerotic acid.

The term “hydroxy fatty acid” is also well acknowledged in the art and includes for example 2-hydroxyhexanoic acid, 2-hydroxyoctanoic acid, 2-hydroxydecanoic acid, 2-hydroxy-dodecanoic acid, β-hydroxylauric acid, 2-hydroxytetradecanoic acid, β-hydroxymyristic acid, 15-hydroxypentadecanoic acid, 16-hydroxyhexadecanoic acid, β-hydroxypalmitic acid, 12-hydroxyoctadecanoic acid, α-hydroxystearic acid, and α-hydroxyarachidic acid.

The fatty acids and the hydroxy fatty acids are preferably saturated.

When the waxy material is or comprises a diglyceride or a triglyceride, the fatty acids, hydroxy fatty acids and aromatic acids, respectively, may be identical or different.

According to this embodiment of the invention, the waxy material is preferably a hard fat (adeps solidus) in accordance with Ph. Eur.

Preferably, the waxy material is a monoglyceride, diglyceride, triglyceride or a mixture thereof, selected from the group consisting of hydrogenated soybean oil, hydrogenated palm oil, hydrogenated castor oil, hydrogenated cottonseed oil, and mixtures thereof.

When the waxy material is or comprises an ester of a fatty acid with a fatty alcohol, the fatty acid is preferably a saturated fatty acid. Preferred examples of fatty acids are already mentioned above in connection with the glycerides. The fatty alcohol is preferably derived from a fatty acid and preferably also saturated.

Preferred representatives of esters of fatty acids with fatty alcohols include but are not limited to natural waxes such as beeswax, carnaubawax, cetyl palmitate, oleyl oleate, spermaceti (cetaceum), candelilla wax, ouricury wax, sugarcane wax, and retamo wax.

When the waxy material is or comprises a paraffin, the paraffin is preferably a hard paraffin (paraffinum solidum, ceresin, zeresin) in accordance with Ph. Eur.

The waxy material may comprise a single waxy material, or a mixture (blend) of different waxy materials, such as two, three, four or five waxy materials, each of which preferably being selected from the group consisting of glycerides, especially monoglycerides, diglycerides, triglycerides; esters of fatty acids with fatty alcohols; and paraffins.

Waxy materials that are suitable for use in the pharmaceutical dosage forms according to the invention are commercially available, e.g. Cera alba, Cera flava, Kolliwax™ HCO, Dynasan® 118, Compritol® 888 ATO, Precirol® ATO 5, Gelucire® 44/14. For details concerning the properties of these products, it can be referred to e.g. the product specification.

Preferred polyalkylene glycols include but are not limited to polymethylene oxide, polyethylene oxide, polypropylene oxide, and the copolymers and mixtures thereof. For the purpose of the specification, a polyalkylene glycol has a molecular weight of up to 20,000 g/mol whereas a polyalkylene oxide has a molecular weight of more than 20,000 g/mol.

In a preferred embodiment, the polyalkylene glycol has a weight average molecular weight (M_{W}) or viscosity average molecular weight (M_{η}) in the range of 1,000 g/mol to 18000 g/mol, and most preferably in the range of 5,000 g/mol to 8,000 g/mol. Suitable methods to determine M_{W} and M_{η} are known to a person skilled in the art. M_{η} is preferably determined by rheological measurements, whereas M_{W} can be determined by gel permeation chromatography (GPC).

Preferred celluloses and cellulose derivatives include but are not limited to microcrystalline cellulose, cellulose esters and cellulose ethers.

Preferred cellulose ethers include nonionic cellulose ethers such as methylcellulose, ethylcellulose, propylcellulose, hydroxymethylcellulose, hydroxyethylcellulose, hydroxypropylcellulose, and hydroxypropylmethylcellulose; as well as ionic cellulose ethers, i.e. cationic cellulose ethers or anionic cellulose ethers such as carboxymethyl cellulose.

In view of their good solubility in aqueous ethanol, however, ethylcellulose and propylcellulose are preferably only contained in comparatively low amounts (preferably at most 1.0 wt.-%) or not contained at all in the pharmaceutical dosage form according to the invention.

Preferred xanthan gums include but are not limited to Grindsted® Xanthan 80 Pharma available from Danisco and CEROGA Xanthan Gum Type 602 available from Roeper.

Suitable xanthan gums which are commercially available include XANTURAL® 75, XANTURAL® 180 and XANTURAL® 11K from CP Kelco; VANZAN® NF, VANZAN® NF-F, VANZAN® NF-C from Vanderbilt Minerals; Haixan® PM80, Haixan® PM200, Haixan® PM40 from Zibo Hailan Chemical Co.; Xanthan Gum Pharmaceutical Grade PHARM200 from ICD Biochemistry Co. and Xanthan Gum from Jungbunzlauer.

Alternatively or additionally, the additional prolonged release matrix material may comprise one or more polymers, preferably selected from the group consisting of polyethylene, polypropylene, polyvinyl chloride, polycarbonate, polystyrene, polyvinylpyrrolidone, poly(alk)acrylate, poly(hydroxy fatty acids), such as for example poly(3-hydroxybutyrate-co-3-hydroxyvalerate) (Biopol®), poly(hydroxyvaleric acid), polycaprolactone, polyvinyl caprolactam, polyesteramide, polyethylene succinate, polylactone, polyglycolide, polyurethane, polyamide, polylactide, polyacetal (for example polysaccharides optionally with modified side chains), polylactide/glycolide, polylactone, polyglycolide, polyorthoester, polyanhydride, block polymers of polyethylene glycol and polybutylene terephthalate (Polyactive), polyanhydride (Polifeprosan), copolymers thereof, block-copolymers thereof (e.g., Poloxamer®), and mixtures of at least two of the stated polymers, or other polymers with the above characteristics.

The pharmaceutical dosage form or, when it is multiparticulate, the particles according to the invention which contain the pharmacologically active ingredient may contain additional pharmaceutical excipients conventionally contained in pharmaceutical dosage forms in conventional amounts, such as antioxidants, preservatives, lubricants, plasticizer, fillers, binders, and the like.

The skilled person will readily be able to determine appropriate further excipients as well as the quantities of each of these excipients. Specific examples of pharmaceutically acceptable carriers and excipients are described in the Handbook of Pharmaceutical Excipients, American Pharmaceutical Association (1986).

In a preferred embodiment, the pharmaceutical dosage form or, when it is multiparticulate, the particles according to the invention which contain the pharmacologically active ingredient do not contain a disintegrant. According to this embodiment, the pharmaceutical dosage form or, when it is multiparticulate, the particles according to the invention which contain the pharmacologically active ingredient preferably do not contain sodium starch glycolate.

Preferably, the pharmaceutical dosage form or, when it is multiparticulate, the particles according to the invention which contain the pharmacologically active ingredient further comprise an antioxidant. Suitable antioxidants include ascorbic acid, butylated hydroxyanisole (BHA), butylated hydroxytoluene (BHT), salts of ascorbic acid, monothioglycerol, phosphorous acid, vitamin C, vitamin E and the derivatives thereof, coniferyl benzoate, nordihydroguajaretic acid, gallus acid esters, sodium bisulfite, particularly preferably butylhydroxytoluene or butylhydroxyanisole and α-tocopherol. The antioxidant is preferably present in quantities of 0.01 wt.-% to 10 wt.-%, more preferably of 0.03 wt.-% to 5 wt.-%, most preferably of 0.05 wt.-% to 2.5 wt.-%, based on the total weight of the pharmaceutical dosage form and the particles, respectively.

In a preferred embodiment, the pharmaceutical dosage form or, when it is multiparticulate, the particles according to the invention which contain the pharmacologically active ingredient further comprise an acid, preferably citric acid. The amount of acid is preferably in the range of 0.01 wt.-% to 20 wt.-%, more preferably in the range of 0.02 wt.-% to 10 wt.-%, and still more preferably in the range of 0.05 wt.-% to 5 wt.-%, and most preferably in the range of 0.1 wt.-% to 1.0 wt.-%, based on the total weight of the pharmaceutical dosage form and the particles, respectively.

In a preferred embodiment, the pharmaceutical dosage form or, when it is multiparticulate, the particles according to the invention which contain the pharmacologically active ingredient contain at least one lubricant. In another preferred embodiment, the pharmaceutical dosage form or, when it is multiparticulate, the particles according to the invention which contain the pharmacologically active ingredient contain no lubricant.

Especially preferred lubricants are selected from
magnesium stearate and stearic acid;
polyoxyethylene glycerol fatty acid esters, such as mixtures of mono-, di- and triesters of glycerol and di- and monoesters of macrogols having molecular weights within the range of from 200 to 4000 g/mol, e.g., macrogolglycerolcaprylocaprate, macrogolglycerollaurate, macrogolglycerolococoate, macrogolglycerollinoleate, macrogol-20-glycerolmonostearate, macrogol-6-glycerolcaprylocaprate, macrogolglycerololeate; macrogolglycerolstearate, macrogolglycerolhydroxystearate, and macrogolglycerolrizinoleate;
polyglycolyzed glycerides, such as the one known and commercially available under the trade name “Labrasol”;
fatty alcohols that may be linear or branched, such as cetylalcohol, stearylalcohol, cetylstearyl alcohol, 2-octyldodecane-1-ol and 2-hexyldecane-1-ol; and
polyethylene glycols having a molecular weight between 10.000 and 60.000 g/mol.

Preferably, the amount of the lubricant ranges from 0.01 wt.-% to 10 wt.-%, more preferably in the range of 0.05 wt.-% to 7.5 wt.-%, most preferably in the range of 0.1 wt.-% to 5 wt.-%, and in particular in the range of 0.1 wt.-% to 1 wt.-%, based on the total weight of the pharmaceutical dosage form and the particles, respectively.

Preferably, the pharmaceutical dosage form or, when it is multiparticulate, the particles according to the invention which contain the pharmacologically active ingredient further comprise a plasticizer. The plasticizer improves the processability of the prolonged release matrix material and additional prolonged release matrix material, respectively. A preferred plasticizer is polyalkylene glycol, like polyethylene glycol, triacetin, fatty acids, fatty acid esters, waxes and/or microcrystalline waxes. Particularly preferred plasticizers are polyethylene glycols, such as PEG 6000.

Preferably, the content of the plasticizer is within the range of from 0.5 to 30 wt.-%, more preferably 1.0 to 25 wt.-%, still more preferably 2.5 wt.-% to 22.5 wt.-%, yet more preferably 5.0 wt.-% to 20 wt.-%, most preferably 6 to 20 wt.-% and in particular 7 wt.-% to 17.5 wt.-%, based on the total weight of the pharmaceutical dosage form and the particles, respectively.

Plasticizers can sometimes act as a lubricant, and lubricants can sometimes act as a plasticizer.

In a preferred embodiment, the pharmaceutical dosage form according to the invention contains no substances which irritate the nasal passages and/or pharynx, i.e. substances which, when administered via the nasal passages and/or pharynx, bring a physical reaction which is either so unpleasant for the patient that he/she does not wish to or cannot continue administration, for example burning, or physiologically counteracts taking of the corresponding active compound, for example due to increased nasal secretion or sneezing. Further examples of substances which irritate the nasal passages and/or pharynx are those which cause burning, itching, urge to sneeze, increased formation of secretions or a combination of at least two of these stimuli. Corresponding substances and the quantities thereof which are conventionally to be used are known to the person skilled in the art. Some of the substances which irritate the nasal passages and/or pharynx are accordingly based on one or more constituents or one or more plant parts of a hot substance drug. Corresponding hot substance drugs are known per se to the person skilled in the art and are described, for example, in “Pharmazeutische Biologie—Drogen and ihre Inhaltsstoffe” by Prof. Dr. Hildebert Wagner, 2nd., revised edition, Gustav Fischer Verlag, Stuttgart-New York, 1982, pages 82 et seq. The corresponding description is hereby introduced as a reference and is deemed to be part of the disclosure.

The pharmaceutical dosage form according to the invention furthermore preferably contains no antagonists for the pharmacologically active ingredient, preferably no antagonists against psychotropic substances, in particular no antagonists against opioids. Antagonists suitable for a given pharmacologically active ingredient are known to the person skilled in the art and may be present as such or in the form of corresponding derivatives, in particular esters or ethers, or in each case in the form of corresponding physiologically acceptable compounds, in particular in the form of the salts or solvates thereof. The pharmaceutical dosage form according to the invention preferably contains no antagonists selected from among the group comprising naloxone, naltrexone, nalmefene, nalide, nalmexone, nalorphine or naluphine, in each case optionally in the form of a corresponding physiologically acceptable compound, in particular in the form of a base, a salt or solvate; and no neuroleptics, for example a compound selected from among the group comprising haloperidol, promethacine, fluphenazine, perphenazine, levomepromazine, thioridazine, perazine, chlorpromazine, chlorprothixine, zuclopenthixol, flupentixol, prothipendyl, zotepine, benperidol, pipamperone, melperone and bromperidol.

The pharmaceutical dosage form according to the invention furthermore preferably contains no emetic. Emetics are known to the person skilled in the art and may be present as such or in the form of corresponding derivatives, in particular esters or ethers, or in each case in the form of corresponding physiologically acceptable compounds, in particular in the form of the salts or solvates thereof. The pharmaceutical dosage form according to the invention preferably contains no emetic based on one or more constituents of ipecacuanha (ipecac) root, for example based on the constituent emetine, as are, for example, described in “Pharmazeutische Biologie—Drogen and ihre Inhaltsstoffe” by Prof. Dr. Hildebert Wagner, 2nd, revised edition, Gustav Fischer Verlag, Stuttgart, N.Y., 1982. The corresponding literature description is hereby introduced as a reference and is deemed to be part of the disclosure. The pharmaceutical dosage form according to the invention preferably also contains no apomorphine as an emetic.

Finally, the pharmaceutical dosage form according to the invention preferably also contains no bitter substance. Bitter substances and the quantities effective for use may be found in US-2003/0064099 A1, the corresponding disclosure of which should be deemed to be the disclosure of the present application and is hereby introduced as a reference. Examples of bitter substances are aromatic oils, such as peppermint oil, eucalyptus oil, bitter almond oil, menthol, fruit aroma substances, aroma substances from lemons, oranges, limes, grapefruit or mixtures thereof, and/or denatonium benzoate.

The pharmaceutical dosage form according to the invention accordingly preferably contains neither substances which irritate the nasal passages and/or pharynx, nor antagonists for the pharmacologically active ingredient, nor emetics, nor bitter substances.

In a preferred embodiment, the pharmaceutical dosage form provides prolonged release of the pharmacologically active ingredient.

Particularly preferably, the pharmacologically active ingredient is embedded in a prolonged release matrix comprising the polyethylene glycol graft copolymer, wherein the prolonged release matrix provides prolonged release of the pharmacologically active ingredient.

For the purpose of specification “prolonged release” preferably means a product in which the rate of release of active compound from the formulation after administration has been reduced over time, in order to maintain therapeutic activity, to reduce toxic effects, or for some other therapeutic purpose such as reducing the dosing frequency.

Preferably, under physiological conditions the pharmaceutical dosage form according to the invention has released after 30 minutes 0.1 to 75%, after 240 minutes 0.5 to 95%, after 480 minutes 1.0 to 100% and after 720 minutes 2.5 to 100% of the pharmacologically active ingredient. Further preferred release profiles R₁ to R₈ are summarized in the table here below [all data in wt.-% of released pharmacologically active ingredient]:

| time | | R₁ | R₂ | R₃ | R₄ | R₅ | R₆ | R₇ | R₈ |
|---|---|---|---|---|---|---|---|---|---|
| 60 | min | 0-30 | 0-50 | 0-50 | 15-25 | 20-30 | 20-50 | 40-70 | 40-80 |
| 120 | min | 0-40 | 0-75 | 0-75 | 25-40 | 35-50 | 40-75 | 60-95 | 80-100 |
| 240 | min | 3-55 | 3-95 | 10-95 | 40-70 | 55-75 | 60-95 | 80-100 | |
| 480 | min | 10-65 | 10-100 | 35-100 | 60-90 | 80-95 | 80-100 | 90-100 | |
| 720 | min | 20-75 | 20-100 | 55-100 | 70-100 | 90-100 | 90-100 | | |
| 960 | min | 30-88 | 30-100 | 70-100 | >80 | 95-100 | | | |
| 1440 | min | 50-100 | 50-100 | >90 | | | | | |
| 2160 | min | >80 | >80 | | | | | | |

Further preferred release profiles R₉ to R₁₆ are summarized in the table here below [all data in wt.-% of released pharmacologically active ingredient]:

| time | | R₉ | R₁₀ | R₁₁ | R₁₂ | R₁₃ | R₁₄ | R₁₅ | R₁₆ |
|---|---|---|---|---|---|---|---|---|---|
| 30 | min | 17.5 ± 7.5 | 25 ± 15 | 30 ± 15 | 30 ± 15 | 12 ± 10 | 5 ± 4 | 2 ± 1.5 | 50 ± 15 |
| 60 | min | 27.0 ± 8.0 | 35 ± 15 | 38 ± 10 | 38 ± 10 | 18 ± 15 | 6 ± 4 | 3 ± 2 | 70 ± 10 |
| 120 | min | 41.5 ± 9.5 | 43 ± 15 | 48 ± 10 | 48 ± 10 | 20 ± 10 | 8 ± 4 | 4 ± 2 | 88 ± 10 |
| 240 | min | 64.5 ± 12.5 | 60 ± 15 | 55 ± 10 | 60 ± 10 | 32 ± 10 | 9 ± 5 | 5 ± 3 | 90 ± 8 |
| 480 | min | 88.0 ± 12.0 | 83 ± 10 | 63 ± 10 | 68 ± 10 | 50 ± 10 | 10 ± 5 | 6 ± 4 | >95 |
| 720 | min | 96.0 ± 9.0 | 98 ± 2 | 67 ± 10 | 70 ± 10 | 60 ± 10 | 12 ± 5 | 6.5 ± 5 | |
| 840 | min | 97.5 ± 7.5 | >98 | 70 ± 15 | 73 ± 15 | 65 ± 20 | 17 ± 10 | 7 ± 6 | |

Suitable in vitro conditions are known to the skilled artisan. In this regard it can be referred to, e.g., the Eur. Ph. Preferably, the release profile is measured under the following conditions: Paddle apparatus equipped without sinker, 50 rpm, 37±5° C., 900 mL simulated intestinal fluid pH 6.8 (phosphate buffer) or pH 4.5. In a preferred embodiment, the rotational speed of the paddle is increased to 75 rpm.

The dosage form according to the invention contains the pharmacologically active ingredient in a controlled-release matrix comprising the polyethylene glycol graft copolymer, wherein, under in vitro conditions, the release profile of the pharmacologically active ingredient from said matrix comprises at least a time interval during which the release preferably follows a zero order kinetics.

A skilled person knows which requirements need to be satisfied with in order to qualify the in vitro release profile of a pharmaceutical dosage form as being of zero order. Pharmacologically active ingredient dissolution from solid dosage forms has been described by kinetic models in which the dissolved amount of pharmacologically active ingredient (Q) is a function of the test time, t or Q=f(t). Some analytical definitions of the Q(t) function are commonly used, such as zero order, first order, Hixson-Crowell, Weibull, Higuchi, Baker-Lonsdale, Korsmeyer-Peppas and Hopfenberg models. Other release parameters, such as dissolution time (tx %), assay time (tx min), dissolution efficacy (ED), difference factor (f1), similarity factor (f2) and Rescigno index (xi1 and xi2) can be used to characterize pharmacologically active ingredient dissolution/release profiles.

For the purpose of specification the term “zero order kinetics” is preferably defined by the equation W₀−Wₜ=K t, where W₀ is the initial amount of pharmacologically active ingredient in the pharmaceutical dosage form, Wₜ is the amount of pharmacologically active ingredient in the pharmaceutical dosage form at time t and K is a proportionality constant. Dividing this equation by W₀ and simplifying fₜ=K₀ t, where fₜ=1−(Wₜ/W₀) and fₜ represents the fraction of pharmacologically active ingredient dissolved in time t and K₀ the apparent dissolution rate constant or zero order release constant. In this way, a graphic of the pharmacologically active ingredient-dissolved fraction versus time will be linear. This relation can be used to describe the dissolution of several types of modified release pharmaceutical dosage forms, as in the case of matrix tablets with low soluble pharmacologically active ingredients, coated forms, osmotic systems, etc. The pharmaceutical dosage forms following this profile release the same amount of pharmacologically active ingredient by unit of time and it is the ideal method of pharmacologically active ingredient release in order to achieve a pharmacological prolonged action. The following relation can, in a simple way, express this model: Q₁=Q₀+K₀ t, where Qₜ is the amount of pharmacologically active ingredient dissolved in time t, Q₀ is the initial amount of pharmacologically active ingredient in the solution (most times, Q₀=0) and K₀ is the zero order release constant (cf. e.g., P. Costa et al., Eur J Pharm Sci. 2001, 13(2), 123-33).

It is evident to the skilled artisan that in praxis pharmaceutical dosage forms usually do not provide exact zero order release, particularly not over the full length of the release period, i.e. from the very beginning until the release of 100% of the pharmacologically active ingredient that was originally contained in the pharmaceutical dosage form. Rather, in praxis in vitro release profiles can be described with a substantial degree of accuracy by these mathematical models, particularly when not considering the initial phase as well as the end phase of the release.

Preferably, the in vitro release profile of the pharmacologically active ingredient from the pharmaceutical dosage form according to the invention comprises a time interval during which the release follows substantially a zero order kinetics, which time interval is preferably the time needed in order to release 50±5%, more preferably 50±10%, still more preferably 50±15%, yet more preferably 50±20%, even more preferably 50±25%, most preferably 50±30%, and in particular 50±35%, of the pharmacologically active ingredient. For example, the time needed in order to release 50±30% of the pharmacologically active ingredient commences with the release of 20% (e.g. after 2.5 hours) and terminates with the release of 80% (e.g. after 10.5 hours) of the pharmacologically active ingredient. During such time interval, the in vitro release profile of the pharmacologically active ingredient from the pharmaceutical dosage form follows substantially zero order kinetics, i.e. is substantially linear.

In a preferred embodiment, the kinetics for the in vitro release of the pharmacologically active ingredient from the pharmaceutical dosage form is approximated by the equation Mₜ/M₀=k tⁿ where t is time, Mₜ is the amount of the pharmacologically active ingredient which has been released at time t, M₀ is the total amount of the pharmacologically active ingredient originally contained in the dosage form, i.e. before exposing the pharmaceutical dosage form to the release medium, k is a constant, and n is the release kinetics exponent. Preferably, the in vitro release profile of the pharmaceutical dosage form according to the invention provides a curve which defines the retarded release in percent to the time. For a defined time period, preferably from the beginning or from a point in time after the beginning, e.g. from the time where 20% have been released, to the time where 95% of the pharmacologically active ingredient have been released from the dosage form according to the invention, the release profile is substantially linear.

Preferably, the time interval during which the release follows zero order kinetics, e.g. where the second derivative of the graph is substantially linear, is at least 20%, more preferably at least 30%, still more preferably at least 40%, yet more preferably at least 50%, even more preferably at least 60%, most preferably at least 70% and in particular at least 80% of the total release time needed for a release of 95 wt.-% of the pharmacologically active ingredient that was originally contained in the pharmaceutical dosage form.

Preferably, the margins (limits) of “substantially linear” can be assessed based on the second derivative of the curve fitted to the measuring points. Ideally, said second derivative is zero. Preferably, however, a certain degree of deviation is also within the meaning of “substantially linear” according to the invention. Preferably, said deviations from the ideal linear behavior can be quantified by a Chi-square-test, which is known to a person skilled in the art. Preferably, the value determined according to the Chi-square-test is at most 2.5, more preferably at most 1.75, still more preferably at most 1.0, yet more preferably at most 0.75, even more preferably at most 0.5, most preferably at most 0.25, and in particular at most 0.1.

Preferably, the zero-order in vitro release kinetics can adequately be described by Mₜ/M_{∞}=k₀ tⁿ, where Mₜ and M_{∞} are the amounts of drug released at time t and the overall amount released, respectively, n is a release exponent indicative of profile shape, and k₀ is the zero-order release rate constant.

In a preferred embodiment, when fitting the relevant portion of the overall in vitro release profile that shows zero-order release kinetics to the equation Mₜ/M_{∞}=k₀ t (i.e. where n=¹), the correlation coefficient of the fit is preferably at least 0.75, more preferably at least 0.80, still more preferably at least 0.85, yet more preferably at least 0.90, even more preferably at least 0.925, most preferably at least 0.95 and in particular at least 0.975.

In a preferred embodiment, the zero-order release rate constant k₀ is within the range of 0.030±0.028 h⁻¹, more preferably 0.030±0.026 h⁻¹, still more preferably 0.030±0.024 h⁻¹, yet more preferably 0.030±0.020 h⁻¹, even more preferably 0.030±0.015 h⁻¹, most preferably 0.030±0.010 h⁻¹, and in particular 0.030±0.005 h⁻¹. In another preferred embodiment, the zero-order release rate constant k₀ is within the range of 0.040±0.035 h⁻¹, more preferably 0.040±0.030 h⁻¹, still more preferably 0.040±0.025 h⁻¹, yet more preferably 0.040±0.020 h⁻¹, even more preferably 0.040±0.015 h⁻¹, most preferably 0.040±0.010 h⁻¹, and in particular 0.040±0.005 h⁻¹. In still another preferred embodiment, the zero-order release rate constant k₀ is within the range of 0.050±0.035 h⁻¹, more preferably 0.050±0.030 h⁻¹, still more preferably 0.050±0.025 h⁻¹, yet more preferably 0.050±0.020 h⁻¹, even more preferably 0.050±0.015 h⁻¹, most preferably 0.050±0.010 h⁻¹, and in particular 0.050±0.005 h⁻¹. In yet another preferred embodiment, the zero-order release rate constant k₀ is within the range of 0.060±0.035 h⁻¹, more preferably 0.060±0.030 h⁻¹, still more preferably 0.060±0.025 h⁻¹, yet more preferably 0.060±0.020 h⁻¹, even more preferably 0.060±0.015 h⁻¹, most preferably 0.060±0.010 h⁻¹, and in particular 0.060±0.005 h⁻¹. In a further preferred embodiment, the zero-order release rate constant k₀ is within the range of 0.070±0.035 h⁻¹, more preferably 0.070±0.030 h⁻¹, still more preferably 0.070±0.025 h⁻¹, yet more preferably 0.070±0.020 h⁻¹, even more preferably 0.070±0.015 h⁻¹, most preferably 0.070±0.010 h⁻¹, and in particular 0.070±0.005 h⁻¹. In a still further preferred embodiment, the zero-order release rate constant k₀ is within the range of 0.080±0.035 h⁻¹, more preferably 0.080±0.030 h⁻¹, still more preferably 0.080±0.025 h⁻¹, yet more preferably 0.080±0.020 h⁻¹, even more preferably 0.080±0.015 h⁻¹, most preferably 0.080±0.010 h⁻¹, and in particular 0.080±0.005 h⁻¹. In a yet further preferred embodiment, the zero-order release rate constant k₀ is within the range of 0.090±0.035 h⁻¹, more preferably 0.090±0.030 h⁻¹, still more preferably 0.090±0.025 h⁻¹, yet more preferably 0.090±0.020 even more preferably 0.090±0.015 h⁻¹, most preferably 0.090±0.010 h⁻¹, and in particular 0.090±0.005 h⁻¹. In another preferred embodiment, the zero-order release rate constant k₀ is within the range of 0.100±0.035 h⁻¹, more preferably 0.100±0.030 h⁻¹, still more preferably 0.100±0.025 h⁻¹, yet more preferably 0.100±0.020 h⁻¹, even more preferably 0.100±0.015 h⁻¹, most preferably 0.100±0.010 h⁻¹, and in particular 0.100±0.005 h⁻¹.

In a preferred embodiment, release exponent n is at least 0.65, more preferably at least 0.70, still more preferably at least 0.75, yet more preferably at least 0.80, even more preferably at least 0.85, most preferably at least 0.90 and in particular at least 0.95.

The zero-order release kinetics of the pharmaceutical dosage form according to the invention preferably does not rely on a coating that remains intact during the release phase and covers the matrix composition in such a manner that only a specific surface area is subject to erosion. Thus, the surface area of the pharmaceutical dosage form according to the invention from which the active substance is released is preferably not kept substantially constant by means of such a coating. On the contrary, the zero-order release kinetics of the pharmaceutical dosage form according to the invention is preferably based on the properties of the matrix in which the pharmacologically active ingredient is embedded so that inert coatings can be completely omitted. Thus, while the pharmaceutical dosage form according to the invention may be coated with conventional coating materials such as polyvinyl caprolactam, it is preferably not coated with inert coating materials that serve the purpose of permanently covering a substantial portion of the outer surface of the dosage form in order to allow drug release only through a predetermined, uncoated portion. Thus, in a preferred embodiment, the pharmaceutical dosage form according to the invention is uncoated, or it is coated with a coating material that substantially covers the complete outer surface of the dosage form, but does not leave a certain portion uncoated.

Preferably, the release profile, the pharmacologically active ingredient, the polyethylene glycol graft copolymer, the optionally present additional prolonged release matrix material and the optionally present pharmaceutical excipients of the pharmaceutical dosage form according to the invention are stable upon storage, preferably upon storage at elevated temperature, e.g. 40° C., for 3 months in sealed containers.

In connection with the release profile, the term “stable” preferably means that when comparing the initial release profile with the release profile after storage, at any given time point the release profiles deviate from one another by not more than 20%, more preferably not more than 15%, still more preferably not more than 10%, yet more preferably not more than 7.5%, most preferably not more than 5.0% and in particular not more than 2.5%.

In connection with the pharmacologically active ingredient, the polyethylene glycol graft copolymer, the optionally present additional prolonged release matrix material and the optional pharmaceutical excipients, the term “stable” preferably means that the pharmaceutical dosage forms satisfy the requirements of EMEA concerning shelf-life of pharmaceutical products.

In a preferred embodiment, the additional prolonged release matrix material exerts an influence on the release profile of the pharmacologically active ingredient. According to this embodiment, a pharmaceutical dosage form according to the invention comprising a pharmacologically active ingredient, a polyethylene glycol graft copolymer and an additional prolonged release matrix material preferably exhibits an increased release rate of the pharmacologically active ingredient than a pharmaceutical dosage form comprising the same types and amounts of the pharmacologically active ingredient and the polyethylene glycol graft copolymer but not containing any additional prolonged release matrix material.

In a preferred embodiment, the pharmaceutical dosage form according to the invention is adapted for administration once daily. In another preferred embodiment, the pharmaceutical dosage form according to the invention is adapted for administration twice daily. In still another preferred embodiment, the pharmaceutical dosage form according to the invention is adapted for administration thrice daily. In yet another preferred embodiment, the pharmaceutical dosage form according to the invention is adapted for administration more frequently than thrice daily, for example 4 times daily, 5 times daily, 6 times daily, 7 times daily or 8 times daily.

For the purpose of the specification, “twice daily” means equal or nearly equal time intervals, i.e., every 12 hours, or different time intervals, e.g., 8 and 16 hours or 10 and 14 hours, between the individual administrations.

For the purpose of the specification, “thrice daily” means equal or nearly equal time intervals, i.e., every 8 hours, or different time intervals, e.g., 6, 6 and 12 hours; or 7, 7 and 10 hours, between the individual administrations.

The pharmaceutical dosage form according to the invention preferably provides tamper resistance in terms of resistance against solvent extraction, and/or resistance against grinding, and/or resistance against dose-dumping in aqueous ethanol.

Preferably, the prolonged release matrix of the pharmaceutical dosage form according to the invention not only provides prolonged release of the pharmacologically active ingredient, but additionally provides tamper resistance, i.e. resistance against solvent extraction, resistance against grinding, and resistance against dose-dumping in aqueous ethanol.

As used herein, the term “tamper resistant” refers to pharmaceutical dosage forms that are resistant to conversion into a form suitable for misuse or abuse by conventional means, particular for nasal and/or intravenous administration.

In this regard, when the pharmaceutical dosage form is multiparticulate, as such it may be crushable by conventional means such as grinding in a mortar or crushing by means of a hammer. However, when the pharmaceutical dosage form is multiparticulate, the particles which contain the pharmacologically active ingredient exhibit mechanical properties such that they cannot be pulverized by conventional means any further. As the particles are of macroscopic size and contain the pharmacologically active ingredient, they cannot be administered nasally thereby rendering the pharmaceutical dosage form tamper resistant.

Further, when trying to disrupt the pharmaceutical dosage forms by means of a hammer or mortar, the particles tend to adhere to one another thereby forming aggregates and agglomerates, respectively, which are larger in size than the untreated particles.

The pharmaceutical dosage form according to the invention preferably exhibits resistance against solvent extraction. Preferably, the prolonged release matrix provides the pharmaceutical dosage form according to the invention with resistance against solvent extraction.

Preferably, when trying to tamper the pharmaceutical dosage form in order to prepare a formulation suitable for abuse by intravenous administration, the liquid part of the formulation that can be separated from the remainder by means of a syringe at room temperature is as less as possible, preferably it contains not more than 75 or 45 or 40 wt.-%, more preferably not more than 35 wt.-%, still more preferably not more than 30 wt.-%, yet more preferably not more than 25 wt.-%, even more preferably not more than 20 wt.-%, most preferably not more than 15 wt.-% and in particular not more than 10 wt.-% of the originally contained pharmacologically active ingredient.

Preferably, this property is tested by (i) dispensing a pharmaceutical dosage form that is either intact or has been manually comminuted by means of two spoons in 5 ml of solvent, either purified water or aqueous ethanol (40 vol. %), (ii) allowing the dispersion to stand for 10 min at room temperature, (iii) drawing up the hot liquid into a syringe (needle 21 G equipped with a cigarette filter), and (iv) determining the amount of the pharmacologically active ingredient contained in the liquid within the syringe.

The pharmaceutical dosage form according to the invention preferably exhibits resistance against grinding. Preferably, the prolonged release matrix provides the pharmaceutical dosage form according to the invention with resistance against grinding.

Preferably, when a pharmaceutical dosage form according to the invention is treated with a commercial coffee mill, preferably type Bosch MKM6000, 180 W, Typ KM13 for 2 minutes, 42±17.5 wt.-%, more preferably 42±15 wt.-%, still more preferably 42±12.5 wt.-%, yet more preferably 42±10 wt.-%, even more preferably 42±7.5 wt.-%, most preferably 42±5 wt.-%, and in particular 42±2.5 wt.-%, of the total weight of the thus obtained material does not pass a sieve having a mesh size of 1.000 mm.

Preferably, when a pharmaceutical dosage form according to the invention is treated with a commercial coffee mill, preferably type Bosch MKM6000, 180 W, Typ KM13, for 2 minutes, 57±17.5 wt.-%, more preferably 57±15 wt.-%, still more preferably 57±12.5 wt.-%, yet more preferably 57±10 wt.-%, even more preferably 57±7.5 wt.-%, most preferably 57±5 wt.-%, and in particular 57±2.5 wt.-%, of the total weight of the thus obtained material does not pass a sieve having a mesh size of 1.000 mm.

Preferably, when a pharmaceutical dosage form according to the invention is treated with a commercial coffee mill, preferably type Bosch MKM6000, 180 W, Typ KM13, for 2 minutes, at least 50 wt.-%, more preferably at least 55 wt.-%, still more preferably at least 60 wt.-%, yet more preferably at least 65 wt.-%, even more preferably at least 70 wt.-%, most preferably at least 75 wt.-%, and in particular at least 80 wt.-%, of the total weight of the thus obtained material does not pass a sieve having a mesh size of 1.000 mm.

Particle size distributions of the ground pharmaceutical dosage form are preferably determined by sieve analysis.

In a preferred embodiment, more than 55%, more preferably more than 60%, still more preferably more than 65%, yet more preferably more than 70%, most preferably 75% and in particular more than 80% of the particles of the ground pharmaceutical dosage form have a size in the range of from 0.2 to 3.3 nm, more preferably of from 0.4 to 3.1 nm, most preferably of from 0.6 to 2.9 and in particular of from 0.7 to 2.8 nm.

Preferred particle distributions P₁ to P₄ are summarized in the table below:

| particle size | amount in % | | | |
|---|---|---|---|---|
| [nm] | P₁ | P₂ | P₃ | P₄ |
| <0.045 | 0.5 ± 0.4 | 0.1 ± 0.09 | 0.3 ± 0.29 | 0.3 ± 0.29 |
| 0.045-0.063 | 0.5 ± 0.4 | 0.3 ± 0.29 | 0.3 ± 0.29 | 0.3 ± 0.29 |
| 0.063-0.090 | 0.5 ± 0.4 | 0.3 ± 0.29 | 0.3 ± 0.29 | 1.0 ± 0.9 |
| 0.090-0.125 | 0.5 ± 0.4 | 0.3 ± 0.29 | 0.3 ± 0.29 | 1.0 ± 0.9 |
| 0.125-0.180 | 0.5 ± 0.4 | 3.0 ± 2.9 | 2.0 ± 1.5 | 2.0 ± 1.5 |
| 0.180-0.250 | 1.5 ± 1.4 | 1.0 ± 0.8 | 2.0 ± 1.5 | 1.0 ± 0.9 |
| 0.250-0.355 | 4.0 ± 3.5 | 5.0 ± 4.0 | 4.0 ± 3.5 | 3.5 ± 2.5 |
| 0.355-0.500 | 7.0 ± 6.0 | 5.0 ± 4.0 | 6.0 ± 4.5 | 7.0 ± 6.0 |
| 0.500-0.710 | 11.0 ± 8.0 | 9.0 ± 7.0 | 11.0 ± 8.0 | 10.0 ± 7.0 |
| 0.710-1.000 | 15.0 ± 12.0 | 10.0 ± 7.0 | 17.0 ± 14.0 | 18.0 ± 15.0 |
| 1.000-1.400 | 20.0 ± 17.0 | 18.0 ± 15.0 | 23.0 ± 20.0 | 28.0 ± 25.0 |
| 1.400-2.000 | 23.0 ± 20.0 | 19.0 ± 16.0 | 12.0 ± 9.0 | 18.0 ± 15.0 |
| 2.000-2.800 | 13.0 ± 10.0 | 16.0 ± 13.0 | 13.0 ± 10.0 | 11.0 ± 8.0 |
| 2.800-4.000 | 1.0 ± 0.8 | 14.0 ± 11.0 | 12.0 ± 9.0 | 0.3 ± 0.29 |
| >4.00 | 0.5 ± 0.45 | 0.3 ± 0.29 | 0.3 ± 0.29 | 0.5 ± 0.45 |

Further preferred particle distributions P₅ to P₈ are summarized in the table below:

| particle size | amount in % | | | |
|---|---|---|---|---|
| [nm] | P₅ | P₆ | P₇ | P₈ |
| <0.045 | 0.3 ± 0.29 | 0.3 ± 0.29 | 0.3 ± 0.29 | 0.3 ± 0.29 |
| 0.045-0.063 | 0.3 ± 0.29 | 0.3 ± 0.29 | 1.0 ± 0.9 | 0.3 ± 0.29 |
| 0.063-0.090 | 0.3 ± 0.29 | 0.3 ± 0.29 | 1.5 ± 1.0 | 0.3 ± 0.29 |
| 0.090-0.125 | 0.3 ± 0.29 | 1.0 ± 0.9 | 3.5 ± 3.0 | 0.3 ± 0.29 |
| 0.125-0.180 | 1.0 ± 0.9 | 1.0 ± 0.9 | 1.0 ± 0.9 | 3.0 ± 2.9 |
| 0.180-0.250 | 2.0 ± 1.5 | 1.0 ± 0.9 | 0.3 ± 0.29 | 1.5 ± 1.0 |
| 0.250-0.355 | 5.0 ± 4.0 | 3.0 ± 2.9 | 0.3 ± 0.29 | 2.0 ± 1.9 |
| 0.355-0.500 | 7.0 ± 6.0 | 7.0 ± 6.0 | 5.0 ± 4.0 | 1.0 ± 0.9 |
| 0.500-0.710 | 13.0 ± 10.0 | 9.0 ± 7.0 | 8.0 ± 6.0 | 3.5 ± 2.5 |
| 0.710-1.000 | 18.0 ± 15.0 | 13.0 ± 10.0 | 55.0 ± 30.0 | 19.5 ± 15.0 |
| 1.000-1.400 | 25.0 ± 22.0 | 20.0 ± 17.0 | 6.5 ± 5.0 | 70.1 ± 50.0 |
| 1.400-2.000 | 10.0 ± 7.0 | 22.0 ± 19.0 | 13.0 ± 10.0 | 2.0 ± 1.9 |
| 2.000-2.800 | 14.0 ± 11.0 | 12.0 ± 9.0 | 3.0 ± 2.9 | 0.3 ± 0.29 |
| 2.800-4.000 | 4.0 ± 3.5 | 9.0 ± 7.0 | 2.0 ± 1.9 | 0.3 ± 0.29 |
| >4.00 | 0.3 ± 0.29 | 0.5 ± 0.45 | 13.0 ± 10.0 | 1.5 ± 1.0 |

In a preferred embodiment, the pharmaceutical dosage form according to the invention is monolithic and has a breaking strength of at least 200 N or, when the pharmaceutical dosage form according to the invention is multiparticulate, at least a fraction of the individual particles have a breaking strength of at least 200 N.

Preferably, the mechanical properties, particularly the breaking strength, substantially relies on the presence and spatial distribution of the polyethylene glycol graft copolymer (the prolonged release matrix material), although its mere presence does typically not suffice in order to achieve said properties. The advantageous mechanical properties may not automatically be achieved by simply processing pharmacologically active ingredient, polyethylene glycol graft copolymer (the prolonged release matrix material), optionally additional prolonged release matrix material, and optionally further excipients by means of conventional methods for the preparation of pharmaceutical dosage forms. In fact, usually suitable apparatuses must be selected for the preparation and critical processing parameters must be adjusted, particularly pressure/force, temperature and time. Thus, even if conventional apparatuses are used, the process protocols usually must be adapted in order to meet the required criteria.

In general, the desired properties may be obtained only if, during preparation of the pharmaceutical dosage form,
suitable components
in suitable amounts
   are exposed to
a sufficient pressure
at a sufficient temperature
for a sufficient period of time.

Thus, regardless of the apparatus used, the process protocols must be adapted in order to meet the required criteria. Therefore, the breaking strength is separable from the composition.

The pharmaceutical dosage form or, when it is multiparticulate, the particles according to the invention which contain the pharmacologically active ingredient preferably have a breaking strength of at least 200 N, at least 210 N, or at least 220 N, preferably at least 230 N, more preferably at least 240 N, still more preferably at least 250 N, yet more preferably at least 260 N, most preferably at least 270 N and in particular at least 300 N, or at least 310 N, at least 320 N.

The “breaking strength” (resistance to crushing) of a pharmaceutical dosage form and of a particle is known to the skilled person. In this regard it can be referred to, e.g., W. A. Ritschel, Die Tablette, 2. Auflage, Editio Cantor Verlag Aulendorf, 2002; H Liebermann et al., Pharmaceutical dosage forms: Pharmaceutical dosage forms, Vol. 2, Informa Healthcare; 2 edition, 1990; and Encyclopedia of Pharmaceutical Technology, Informa Healthcare; 1 edition.

For the purpose of specification, the breaking strength is preferably defined as the amount of force that is necessary in order to fracture a pharmaceutical dosage form and a particle, respectively (=breaking force). Therefore, for the purpose of specification, a pharmaceutical dosage form and a particle, respectively, does preferably not exhibit the desired breaking strength when it breaks, i.e., is fractured into at least two independent parts that are separated from one another. In another preferred embodiment, however, the pharmaceutical dosage form and particle, respectively, is regarded as being broken if the force decreases by 25% (threshold value) of the highest force measured during the measurement (see below).

The pharmaceutical dosage forms and particles, respectively, according to the invention are distinguished from conventional pharmaceutical dosage forms and particles, respectively, in that due to their breaking strength, they cannot be pulverized by the application of force with conventional means, such as for example a pestle and mortar, a hammer, a mallet or other usual means for pulverization, in particular devices developed for this purpose (pharmaceutical dosage form crushers). In this regard “pulverization” means crumbling into small particles. Avoidance of pulverization virtually rules out oral or parenteral, in particular intravenous or nasal abuse.

Conventional pharmaceutical dosage forms and particles, respectively, typically have a breaking strength well below 200 N.

The breaking strength of conventional round pharmaceutical dosage forms/particles may be estimated according to the following empirical formula:

Breaking Strength [in N]=10×Diameter of pharmaceutical dosage form/particle [in mm].

Thus, according to said empirical formula, a round pharmaceutical dosage form/particle having a breaking strength of at least 300 N would require a diameter of at least 30 mm. Such a particle, however, could not be swallowed, let alone a pharmaceutical dosage form containing a plurality of such particles. The above empirical formula preferably does not apply to the pharmaceutical dosage form and particles, respectively, according to the invention, which are not conventional but rather special.

Further, the actual mean chewing force is 220 N (cf., e.g., P. A. Proeschel et al., J Dent Res, 2002, 81(7), 464-468). This means that conventional pharmaceutical dosage forms and particle, respectively, having a breaking strength well below 200 N may be crushed upon spontaneous chewing, whereas the pharmaceutical dosage forms and particles, respectively, according to the invention may preferably not.

Still further, when applying a gravitational acceleration of 9.81 m/s², 200 N correspond to a gravitational force of more than 20 kg, i.e. the pharmaceutical dosage form and particle, respectively, according to the invention can preferably withstand a weight of more than 20 kg without being pulverized.

Methods for measuring the breaking strength are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Pharmaceutical dosage forms”. The particles may be subjected to the same or similar breaking strength test as the pharmaceutical dosage form. The test is intended to determine, under defined conditions, the resistance to crushing of pharmaceutical dosage forms and individual particles, respectively, measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the pharmaceutical dosage form and individual particle, respectively. The apparatus is calibrated using a system with a precision of 1 Newton. The pharmaceutical dosage form and particle, respectively, is placed between the jaws, taking into account, where applicable, the shape, the break-mark and the inscription; for each measurement the pharmaceutical dosage form and particle, respectively, is oriented in the same way with respect to the direction of application of the force (and the direction of extension in which the breaking strength is to be measured). The measurement is carried out on 10 pharmaceutical dosage forms and particles, respectively, taking care that all fragments have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a pharmaceutical dosage form and particle, respectively, to fail (i.e., break) in a specific plane. The pharmaceutical dosage forms and particles, respectively, are generally placed between two platens, one of which moves to apply sufficient force to the pharmaceutical dosage form and particle, respectively, to cause fracture. For conventional, round (circular cross-section) pharmaceutical dosage forms and particles, respectively, loading occurs across their diameter (sometimes referred to as diametral loading), and fracture occurs in the plane. The breaking force of pharmaceutical dosage forms and particles, respectively, is commonly called hardness in the pharmaceutical literature; however, the use of this term is misleading. In material science, the term hardness refers to the resistance of a surface to penetration or indentation by a small probe. The term crushing strength is also frequently used to describe the resistance of pharmaceutical dosage forms and particle, respectively, to the application of a compressive load. Although this term describes the true nature of the test more accurately than does hardness, it implies that pharmaceutical dosage forms and particles, respectively, are actually crushed during the test, which is often not the case.

Alternatively, the breaking strength (resistance to crushing) can be measured in accordance with WO 2008/107149, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturer's test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S06 for the centring device.

In a preferred embodiment, the pharmaceutical dosage form and particle, respectively, is regarded as being broken if it is fractured into at least two separate pieces.

The pharmaceutical dosage form and particle, respectively, according to the invention preferably exhibit mechanical strength over a wide temperature range, in addition to the breaking strength (resistance to crushing) optionally also sufficient hardness, impact resistance, impact elasticity, tensile strength and/or modulus of elasticity, optionally also at low temperatures (e.g. below −24° C., below −40° C. or possibly even in liquid nitrogen), for it to be virtually impossible to pulverize by spontaneous chewing, grinding in a mortar, pounding, etc. Thus, preferably, the comparatively high breaking strength of the pharmaceutical dosage form and particle, respectively, according to the invention is maintained even at low or very low temperatures, e.g., when the pharmaceutical dosage form is initially chilled to increase its brittleness, for example to temperatures below −25° C., below −40° C. or even in liquid nitrogen.

The pharmaceutical dosage form and particle, respectively, according to the invention is characterized by a certain degree of breaking strength. This does not mean that it must also exhibit a certain degree of hardness. Hardness and breaking strength are different physical properties. Therefore, the tamper resistance of the pharmaceutical dosage form does not necessarily depend on the hardness of the pharmaceutical dosage form and particle, respectively. For instance, due to its breaking strength, impact strength, elasticity modulus and tensile strength, respectively, the pharmaceutical dosage form and particle, respectively, can preferably be deformed, e.g. plastically, when exerting an external force, for example using a hammer, but cannot be pulverized, i.e., crumbled into a high number of fragments. In other words, the pharmaceutical dosage form and particle, respectively, according to the invention are characterized by a certain degree of breaking strength, but not necessarily also by a certain degree of form stability.

Therefore, in the meaning of the specification, a pharmaceutical dosage form and particle, respectively, that is deformed when being exposed to a force in a particular direction of extension but that does not break (plastic deformation or plastic flow) is preferably to be regarded as having the desired breaking strength in said direction of extension.

Preferred pharmaceutical dosage forms and particles, respectively, are those having a suitable tensile strength as determined by a test method currently accepted in the art. Further preferred pharmaceutical dosage forms and particles, respectively, are those having a Young's Modulus as determined by a test method of the art. Still further preferred pharmaceutical dosages form and particles, respectively, are those having an acceptable elongation at break.

The pharmaceutical dosage form according to the invention preferably exhibits resistance against dose-dumping in aqueous ethanol. Preferably, the prolonged release matrix provides the pharmaceutical dosage form according to the invention with resistance against dose-dumping in aqueous ethanol.

The pharmaceutical dosage form can be tested in vitro using ethanol/simulated gastric fluid of 0%, 20% and 40% to evaluate alcohol extractability. Testing is preferably performed using standard procedures, e.g. USP Apparatus 1 (basket) or USP Apparatus 2 (paddle) at e.g. 50 rpm or 75 rpm in e.g. 500 ml of media at 37° C., using a Perkin Elmer UV/VIS Spectrometer Lambda 20, UV at an appropriate wavelength for detection of the pharmacologically active ingredient present therein. Sample time points preferably include 0.5 and 1 hour.

Preferably, when comparing the in vitro release profile at 37° C. in simulated gastric fluid with the in vitro release profile in ethanol/simulated gastric fluid (40 vol.-%) at 37° C., the in vitro release in ethanol/simulated gastric fluid (40 vol.-%) is preferably not substantially accelerated compared to the in vitro release in simulated gastric fluid. Preferably, in this regard “substantially” means that at any given time point the in vitro release in ethanol/simulated gastric fluid (40 vol.-%) relatively deviates from the in vitro release in simulated gastric fluid by not more than +25%, more preferably not more than +20%, still more preferably not more than +15%, yet more preferably not more than +10%, even more preferably not more than +7.5%, most preferably not more than +5.0% and in particular not more than +2.5%.

A substantial relative acceleration of the in vitro release in ethanol/simulated gastric fluid (40 vol.-%) compared to the in vitro release in simulated gastric fluid is to be prevented according to the invention. However, a substantial relative deceleration of the in vitro release in ethanol/simulated gastric fluid (40 vol.-%) compared to the in vitro release in simulated gastric fluid, e.g., a relative deviation by −25% or more, may be possible and can even be desirable.

The pharmacologically active ingredient having psychotropic action is not particularly limited.

For the purpose of definition, a pharmacologically active ingredient having psychotropic action is preferably meant to refer to any pharmacologically active ingredient which crosses the blood-brain barrier and acts primarily upon the central nervous system where it affects brain function, resulting in alterations in perception, mood, consciousness, cognition, and behavior.

In a preferred embodiment, the pharmaceutical dosage form contains only a single pharmacologically active ingredient. In another preferred embodiment, the pharmaceutical dosage form contains a combination of two or more pharmacologically active ingredients.

Preferably, the pharmaceutical dosage form according to the invention comprises a pharmacologically active ingredient having potential for abuse and potential for dose dumping in ethanol. Active ingredients with potential for being abused are known to the person skilled in the art and comprise e.g. tranquilizers, stimulants, barbiturates, narcotics, opioids or opioid derivatives.

Preferably, the pharmacologically active ingredient is selected from the group consisting of opiates, opioids, stimulants, tranquilizers, other narcotics and anesthetics. Preferably, the pharmacologically active ingredient is selected from the group consisting of ethers; halogenated hydrocarbons; pain barbiturates; and barbiturates in combination with other drugs; opioid anesthetics; or any other general anesthetics.

In a particularly preferred embodiment, the pharmacologically active ingredient is an opioid or a physiologically acceptable salt thereof.

According to the ATC index, opioids are divided into natural opium alkaloids, phenylpiperidine derivatives, diphenylpropylamine derivatives, benzomorphan derivatives, oripavine derivatives, morphinan derivatives and others.

The following opiates, opioids, tranquilizers, anesthetics or other narcotics are substances with a psychotropic action, i.e. have a potential of abuse, and hence are preferably contained in the pharmaceutical dosage form and the particles, respectively: alfentanil, allobarbital, allylprodine, alphaprodine, alprazolam, amfepramone, amphetamine, amphetaminil, amobarbital, anileridine, apocodeine, axomadol, barbital, bemidone, benzylmorphine, bezitramide, bromazepam, brotizolam, buprenorphine, butobarbital, butorphanol, camazepam, carfentanil, cathine/D-norpseudoephedrine, chlordiazepoxide, clobazam clofedanol, clonazepam, clonitazene, clorazepate, clotiazepam, cloxazolam, cocaine, codeine, cyclobarbital, cyclorphan, cyprenorphine, delorazepam, desomorphine, dextromoramide, dextropropoxyphene, dezocine, diampromide, diamorphone, diazepam, dihydrocodeine, dihydromorphine, dihydromorphone, dimenoxadol, dimephetamol, dimethylthiambutene, dioxaphetylbutyrate, dipipanone, dronabinol, eptazocine, estazolam, ethoheptazine, ethylmethylthiambutene, ethyl loflazepate, ethylmorphine, etonitazene, etorphine, faxeladol, fencamfamine, fenethylline, fenpipramide, fenproporex, fentanyl, fludiazepam, flunitrazepam, flurazepam, halazepam, haloxazolam, heroin, hydrocodone, hydromorphone, hydroxypethidine, isomethadone, hydroxymethylmorphinan, ketamine, (S)-ketamine, ketazolam, ketobemidone, levacetylmethadol (LAAM), levomethadone, levorphanol, levophenacylmorphane, levoxemacin, lisdexamfetamine dimesylate, lofentanil, loprazolam, lorazepam, lormetazepam, mazindol, medazepam, mefenorex, meperidine, meprobamate, metapon, meptazinol, metazocine, methylmorphine, metamphetamine, methadone, metha-qualone, 3-methylfentanyl, 4-methylfentanyl, methylphenidate, methylphenobarbital, methyprylon, metopon, midazolam, modafinil, morphine, myrophine, nabilone, nalbuphene, nalorphine, narceine, nicomorphine, nimetazepam, nitrazepam, nordazepam, norlevorphanol, normethadone, normorphine, norpipanone, opium, oxazepam, oxazolam, oxycodone, oxymorphone, Papaver somniferum, papaveretum, pemoline, pentazocine, pentobarbital, pethidine, phenadoxone, phenomorphane, phenazocine, phenoperidine, piminodine, pholcodeine, phenmetrazine, phenobarbital, phentermine, pinazepam, pipradrol, piritramide, prazepam, profadol, proheptazine, promedol, properidine, propoxyphene, remifentanil, secbutabarbital, secobarbital, sufentanil, tapentadol, temazepam, tetrazepam, tilidine (cis and trans), tramadol, triazolam, vinylbital, N-(1-methyl-2-piperidinoethyl)-N-(2-pyridyl)-propionamide, (1R,2R)-3-(3-dimethylamino-1-ethyl-2-methyl-propyl)phenol, (1R,2R,4S)-2-(dimethylamino)methyl-4-(p-fluorobenzyloxy)-1-(m-methoxyphenyl)cyclohexanol, (1R,2R)-3-(2-dimethylaminomethyl-cyclohexyl)phenol, (1S,2S)-3-(3-dimethylamino-1-ethyl-2-methyl-propyl)phenol, (2R,3R)-1-dimethylamino-3 (3-methoxyphenyl)-2-methyl-pentan-3-ol, (1RS,3RS,6RS)-6-dimethylaminomethyl-1-(3-methoxyphenyl)-cyclohexane-1,3-diol, preferably as racemate, 3-(2-dimethylaminomethyl-1-hydroxy-cyclohexyl)phenyl 2-(4-isobutyl-phenyl)propionate, 3-(2-dimethylaminomethyl-1-hydroxy-cyclohexyl)phenyl 2-(6-methoxy-naphthalen-2-yl)propionate, 3-(2-dimethylaminomethyl-cyclohex-1-enyl)-phenyl 2-(4-isobutyl-phenyl)propionate, 3-(2-dimethylaminomethyl-cyclohex-1-enyl)-phenyl 2-(6-methoxy-naphthalen-2-yl)propionate, (RR—SS)-2-acetoxy-4-trifluoromethyl-benzoic acid 3-(2-dimethylaminomethyl-1-hydroxy-cyclohexyl)-phenyl ester, (RR—SS)-2-hydroxy-4-trifluoromethyl-benzoic acid 3-(2-dimethylaminomethyl-1-hydroxy-cyclohexyl)-phenyl ester, (RR—SS)-4-chloro-2-hydroxy-benzoic acid 3-(2-dimethylaminomethyl-1-hydroxy-cyclohexyl)-phenyl ester, (RR—SS)-2-hydroxy-4-methyl-benzoic acid 3-(2-dimethylaminomethyl-1-hydroxy-cyclohexyl)-phenyl ester, (RR—SS)-2-hydroxy-4-methoxy-benzoic acid 3-(2-dimethylaminomethyl-1-hydroxy-cyclohexyl)-phenyl ester, (RR—SS)-2-hydroxy-5-nitro-benzoic acid 3-(2-dimethylaminomethyl-1-hydroxy-cyclohexyl)-phenyl ester, (RR—SS)-2′,4′-difluoro-3-hydroxy-biphenyl-4-carboxylic acid 3-(2-dimethylaminomethyl-1-hydroxy-cyclohexyl)-phenyl ester, and corresponding stereoisomeric compounds, in each case the corresponding derivatives thereof, physiologically acceptable enantiomers, stereoisomers, diastereomers and racemates and the physiologically acceptable derivatives thereof, e.g. ethers, esters or amides, and in each case the physiologically acceptable compounds thereof, in particular the acid or base addition salts thereof and solvates, e.g. hydrochlorides.

In a preferred embodiment, the pharmacologically active ingredient is selected from the group consisting of tramadol, tapentadol, faxeladol and axomadol.

In another preferred embodiment, the pharmacologically active ingredient is selected from the group consisting of DPI-125, M6G (CE-04-410), ADL-5859, CR-665, NRP290 and sebacoyl dinalbuphine ester.

In still another preferred embodiment, the pharmacologically active ingredient is selected from the group consisting of oxycodone, oxymorphone, hydrocodone, hydromorphone, taramdol, tapentadol, morphine, buprenorphine and the physiologically acceptable salts thereof.

In yet another preferred embodiment, the pharmacologically active ingredient is selected from the group consisting of 1,1-(3-dimethylamino-3-phenylpentamethylene)-6-fluoro-1,3,4,9-tetrahydropyrano[3,4-b]indole, particularly its hemicitrate; 1,1-[3-dimethylamino-3-(2-thienyl)pentamethylene]-1,3,4,9-tetrahydropyrano[3,4-b]indole, particularly its citrate; and 1,1-[3-dimethylamino-3-(2-thienyl)pentamethylene]-1,3,4,9-tetrahydropyrano[3,4-b]-6-fluoroindole, particularly its hemicitrate. These compounds are known from, e.g., WO 2004/043967, WO 2005/066183.

The pharmacologically active ingredient may be present in form of a physiologically acceptable salt, e.g. physiologically acceptable acid addition salt.

Physiologically acceptable acid addition salts comprise the acid addition salt forms which can conveniently be obtained by treating the base form of the active ingredient with appropriate organic and inorganic acids. Active ingredients containing an acidic proton may be converted into their non-toxic metal or amine addition salt forms by treatment with appropriate organic and inorganic bases. The term addition salt also comprises the hydrates and solvent addition forms which the active ingredients are able to form. Examples of such forms are e.g. hydrates, alcoholates and the like.

It has been surprisingly found that the content of the pharmacologically active ingredient in the pharmaceutical dosage form and in the particles, respectively, can be optimized in order to provide the best compromise between tamper-resistance, disintegration time and drug release, drug load, processability (especially pharmaceutical dosage formability) and patient compliance.

The pharmacologically active ingredient is present in the pharmaceutical dosage form in a therapeutically effective amount. The amount that constitutes a therapeutically effective amount varies according to the active ingredients being used, the condition being treated, the severity of said condition, the patient being treated, and the frequency of administration.

The content of the pharmacologically active ingredient in the pharmaceutical dosage form is not limited. The dose of the pharmacologically active ingredient which is adapted for administration preferably is in the range of 0.1 mg to 500 mg, more preferably in the range of 1.0 mg to 400 mg, even more preferably in the range of 5.0 mg to 300 mg, and most preferably in the range of 10 mg to 250 mg. In a preferred embodiment, the total amount of the pharmacologically active ingredient that is contained in the pharmaceutical dosage form is within the range of from 0.01 to 200 mg, more preferably 0.1 to 190 mg, still more preferably 1.0 to 180 mg, yet more preferably 1.5 to 160 mg, most preferably 2.0 to 100 mg and in particular 2.5 to 80 mg.

Preferably, the content of the pharmacologically active ingredient is within the range of from 0.01 to 80 wt.-%, more preferably 0.1 to 50 wt.-%, still more preferably 1 to 35 wt.-%, based on the total weight of the pharmaceutical dosage form.

In a preferred embodiment, the content of pharmacologically active ingredient is within the range of from 5.0±4.5 wt.-%, or 7.5±7.0 wt.-%, or 10±9.0 wt.-%, or 12.5±12.0 wt.-%, or 15±14 wt.-%, or 17.5±17.0 wt.-%, or 20±19 wt.-%, or 22.5±22.0 wt.-%, or 25±24 wt.-%; more preferably 5.0±4.0 wt.-%, or 7.5±6.0 wt.-%, or 10±8.0 wt.-%, or 12.5±12.0 wt.-%, or 15±12 wt.-%, or 17.5±15.0 wt.-%, or 20±19 wt.-%, or 22.5±22.0 wt.-%, or 25±24 wt.-%; still more preferably 5.0±3.5 wt.-%, or 7.5±5.0 wt.-%, or 10±7.0 wt.-%, or 12.5±10.0 wt.-%, or 15±10 wt.-%, or 17.5±13.0 wt.-%, or 20±17 wt.-%, or 22.5±19.0 wt.-%, or 25±21 wt.-%; yet more preferably 5.0±3.0 wt.-%, or 7.5±4.0 wt.-%, or 10±6.0 wt.-%, or 12.5±8.0 wt.-%, or 15±8.0 wt.-%, or 17.5±11.0 wt.-%, or 20±15 wt.-%, or 22.5±16.0 wt.-%, or 25±18 wt.-%; even more preferably 5.0±2.5 wt.-%, or 7.5±3.0 wt.-%, or 10±5.0 wt.-%, or 12.5±6.0 wt.-%, or 15±6.0 wt.-%, or 17.5±9.0 wt.-%, or 20±13 wt.-%, or 22.5±13.0 wt.-%, or 25±15 wt.-%; most preferably 5.0±2.0 wt.-%, or 7.5±2.0 wt.-%, or 10±4.0 wt.-%, or 12.5±4.0 wt.-%, or 15±4.0 wt.-%, or 17.5±7.0 wt.-%, or 20±11 wt.-%, or 22.5±10.0 wt.-%, or 25±12 wt.-%; and in particular 5.0±1.5 wt.-%, or 7.5±1.0 wt.-%, or 10±3.0 wt.-%, or 12.5±2.0 wt.-%, or 15±2.0 wt.-%, or 17.5±5.0 wt.-%, or 20±9 wt.-%, or 22.5±7.0 wt.-%, or 25±9 wt.-%; in each case either based on the total weight of the pharmaceutical dosage form or, when the pharmaceutical dosage form is multiparticulate, based on the total weight of the particles that contain the pharmacologically active ingredient.

In another preferred embodiment, the content of pharmacologically active ingredient is within the range of from 20±6 wt.-%, more preferably 20±5 wt.-%, still more preferably 20±4 wt.-%, most preferably 20±3 wt.-%, and in particular 20±2 wt.-%, either based on the total weight of the pharmaceutical dosage form or, when the pharmaceutical dosage form is multiparticulate, based on the total weight of the particles that contain the pharmacologically active ingredient. In still another preferred embodiment, the content of pharmacologically active ingredient is within the range of from 25±6 wt.-%, more preferably 25±5 wt.-%, still more preferably 25±4 wt.-%, most preferably 25±3 wt.-%, and in particular 25±2 wt.-%, either based on the total weight of the pharmaceutical dosage form or, when the pharmaceutical dosage form is multiparticulate, based on the total weight of the particles that contain the pharmacologically active ingredient. In yet another preferred embodiment, the content of pharmacologically active ingredient is within the range of from 30±6 wt.-%, more preferably 30±5 wt.-%, still more preferably 30±4 wt.-%, most preferably 30±3 wt.-%, and in particular 30±2 wt.-%, either based on the total weight of the pharmaceutical dosage form or, when the pharmaceutical dosage form is multiparticulate, based on the total weight of the particles that contain the pharmacologically active ingredient. In even another preferred embodiment, the content of pharmacologically active ingredient is within the range of from 34±6 wt.-%, more preferably 34±5 wt.-%, still more preferably 34±4 wt.-%, most preferably 34±3 wt.-%, and in particular 34±2 wt.-%, either based on the total weight of the pharmaceutical dosage form or, when the pharmaceutical dosage form is multiparticulate, based on the total weight of the particles that contain the pharmacologically active ingredient. In a further preferred embodiment, the content of pharmacologically active ingredient is within the range of from 40±6 wt.-%, more preferably 40±5 wt.-%, still more preferably 40±4 wt.-%, most preferably 40±3 wt.-%, and in particular 40±2 wt.-%, either based on the total weight of the pharmaceutical dosage form or, when the pharmaceutical dosage form is multiparticulate, based on the total weight of the particles that contain the pharmacologically active ingredient.

The skilled person may readily determine an appropriate amount of pharmacologically active ingredient to include in a pharmaceutical dosage form. For instance, in the case of analgesics, the total amount of pharmacologically active ingredient present in the pharmaceutical dosage form is that sufficient to provide analgesia. The total amount of pharmacologically active ingredient administered to a patient in a dose will vary depending on numerous factors including the nature of the pharmacologically active ingredient, the weight of the patient, the severity of the pain, the nature of other therapeutic agents being administered etc.

In a preferred embodiment, the pharmacologically active ingredient is contained in the pharmaceutical dosage form in an amount of 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, or 300±5 mg. In another preferred embodiment, the pharmacologically active ingredient is contained in the pharmaceutical dosage form in an amount of 5±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, 160±2.5 mg, 165±2.5 mg, 170±2.5 mg, 175±2.5 mg, 180±2.5 mg, 185±2.5 mg, 190±2.5 mg, 195±2.5 mg, 200±2.5 mg, 205±2.5 mg, 210±2.5 mg, 215±2.5 mg, 220±2.5 mg, 225±2.5 mg, 230±2.5 mg, 235±2.5 mg, 240±2.5 mg, 245±2.5 mg, 250±2.5 mg, 255±2.5 mg, 260±2.5 mg, or 265±2.5 mg.

In a particularly preferred embodiment, the pharmacologically active ingredient is oxycodone, preferably its HCl salt, and the pharmaceutical dosage form is adapted for administration twice daily. In this embodiment, the pharmacologically active ingredient is preferably contained in the pharmaceutical dosage form in a total amount of from 1 to 80 mg. In another particularly preferred embodiment, the pharmacologically active ingredient is oxycodone, preferably its HCl salt, and the pharmaceutical dosage form is adapted for administration once daily. In this embodiment, the pharmacologically active ingredient is preferably contained in the pharmaceutical dosage form in a total amount of from 2 to 320 mg.

In another particularly preferred embodiment, the pharmacologically active ingredient is oxymorphone, preferably its HCl salt, and the pharmaceutical dosage form is adapted for administration twice daily. In this embodiment, the pharmacologically active ingredient is preferably contained in the pharmaceutical dosage form in a total amount of from 5 to 40 mg. In another particularly preferred embodiment, the pharmacologically active ingredient is oxymorphone, preferably its HCl salt, and the pharmaceutical dosage form is adapted for administration once daily. In this embodiment, the pharmacologically active ingredient is preferably contained in the pharmaceutical dosage form in a total amount of from 10 to 80 mg.

In another particularly preferred embodiment, the pharmacologically active ingredient is tapentadol, preferably its HCl salt, and the pharmaceutical dosage form is adapted for administration once daily or twice daily. In this embodiment, the pharmacologically active ingredient is preferably contained in the pharmaceutical dosage form in a total amount of from 25 to 250 mg.

In still another particularly preferred embodiment, the pharmacologically active ingredient is hydromorphone, preferably its HCl salt, and the pharmaceutical dosage form is adapted for administration twice daily. In this embodiment, the pharmacologically active ingredient is preferably contained in the pharmaceutical dosage form in a total amount of from 2 to 52 mg. In another particularly preferred embodiment, the pharmacologically active ingredient is hydromorphone, preferably its HCl salt, and the pharmaceutical dosage form is adapted for administration once daily. In this embodiment, the pharmacologically active ingredient is preferably contained in the pharmaceutical dosage form in a total amount of from 4 to 104 mg.

In yet another particularly preferred embodiment, the pharmacologically active ingredient is tramadol, preferably its HCl salt, and the pharmaceutical dosage form is adapted for administration twice daily. In this embodiment, the pharmacologically active ingredient is preferably contained in the pharmaceutical dosage form in a total amount of from 5 to 300 mg. In another particularly preferred embodiment, the pharmacologically active ingredient is tramadol, preferably its HCl salt, and the pharmaceutical dosage form is adapted for administration once daily. In this embodiment, the pharmacologically active ingredient is preferably contained in the pharmaceutical dosage form in a total amount of from 10 to 500 mg.

In another particularly preferred embodiment, the pharmacologically active ingredient is hydrocodone, preferably its HCl salt, and the pharmaceutical dosage form is adapted for administration twice daily. In this embodiment, the pharmacologically active ingredient is preferably contained in the pharmaceutical dosage form in a total amount of from 5 to 250 mg. In another particularly preferred embodiment, the pharmacologically active ingredient is hydrocodone, preferably its HCl salt, and the pharmaceutical dosage form is adapted for administration once daily. In this embodiment, the pharmacologically active ingredient is preferably contained in the pharmaceutical dosage form in a total amount of from 5 to 250 mg.

In still another particularly preferred embodiment, the pharmacologically active ingredient is morphine, preferably its HCl or H₂SO₄ salt, and the pharmaceutical dosage form is adapted for administration twice daily. In this embodiment, the pharmacologically active ingredient is preferably contained in the pharmaceutical dosage form in a total amount of from 5 to 250 mg. In another particularly preferred embodiment, the pharmacologically active ingredient is morphine, preferably its HCl or H₂SO₄ salt, and the pharmaceutical dosage form is adapted for administration once daily. In this embodiment, the pharmacologically active ingredient is preferably contained in the pharmaceutical dosage form in a total amount of from 5 to 250 mg.

In another particularly preferred embodiment, the pharmacologically active ingredient is buprenorphine, preferably its HCl salt, and the pharmaceutical dosage form is adapted for administration twice daily. In this embodiment, the pharmacologically active ingredient is preferably contained in the pharmaceutical dosage form in a total amount of from 1 to 12 mg. In another particularly preferred embodiment, the pharmacologically active ingredient is buprenorphine, preferably its HCl salt, and the pharmaceutical dosage form is adapted for administration once daily. In this embodiment, the pharmacologically active ingredient is preferably contained in the pharmaceutical dosage form in a total amount of from 2 to 12 mg.

When the pharmaceutical dosage form is multiparticulate, the particles present in the pharmaceutical dosage forms according to the invention preferably comprise 3 to 75 wt.-% of pharmacologically active ingredient, more preferably 5 to 70 wt.-% of pharmacologically active ingredient, still more preferably 7.5 to 65 wt.-% of pharmacologically active ingredient, based on the total weight of a particle.

When the pharmaceutical dosage form is multiparticulate, the content of the pharmacologically active ingredient is preferably at least 5 wt.-%, more preferably at least 10 wt.-%, still more preferably at least 15 wt.-%, yet more preferably at least 20 wt.-%, most preferably at least 25 wt.-% and in particular at least 30 wt.-%, based on the total weight of a particle.

When the pharmaceutical dosage form is multiparticulate, the content of the pharmacologically active ingredient is preferably at most 70 wt.-%, more preferably at most 65 wt.-%, still more preferably at most 60 wt.-%, yet more preferably at most 55 wt.-%, most preferably at most 50 wt.-%, based on the total weight of a particle.

In a preferred embodiment, when the pharmaceutical dosage form is multiparticulate, the content of the pharmacologically active ingredient is within the range of 35±30 wt.-%, more preferably 35±25 wt.-%, still more preferably 35±20 wt.-%, yet more preferably 35±15 wt.-%, most preferably 35±10 wt.-%, and in particular 35±5 wt.-%, based on the total weight of a particle. In another preferred embodiment, when the pharmaceutical dosage form is multiparticulate, the content of the pharmacologically active ingredient is within the range of 45±30 wt.-%, more preferably 45±25 wt.-%, still more preferably 45±20 wt.-%, yet more preferably 45±15 wt.-%, most preferably 45±10 wt.-%, and in particular 45±5 wt.-%, based on the total weight of a particle. In still another preferred embodiment, when the pharmaceutical dosage form is multiparticulate, the content of the pharmacologically active ingredient is within the range of 55±30 wt.-%, more preferably 55±25 wt.-%, still more preferably 55±20 wt.-%, yet more preferably 55±15 wt.-%, most preferably 55±10 wt.-%, and in particular 55±5 wt.-%, based on the total weight of a particle.

The pharmacologically active ingredient that is included in the preparation of the pharmaceutical dosage forms according to the invention preferably has an average particle size of less than 500 microns, still more preferably less than 300 microns, yet more preferably less than 200 or 100 microns. There is no lower limit on the average particle size and it may be, for example, 50 microns. The particle size of pharmacologically active ingredients may be determined by any technique conventional in the art, e.g. laser light scattering, sieve analysis, light microscopy or image analysis. Generally speaking it is preferable that the largest dimension of the pharmacologically active ingredient particle be less than the size of the particles (e.g. less than the smallest dimension of the particles).

In a preferred embodiment, the pharmaceutical dosage form according to the invention, preferably the particles, comprise an opioid (agonist) as well as an opioid antagonist.

Any conventional opioid antagonist may be present, e.g. naltrexone or naloxone or their pharmaceutically acceptable salts. Naloxone, including its salts, is particularly preferred. The opioid antagonist may be present within the particles or within the matrix. Alternatively, opioid antagonist may be provided in separate particles to the pharmacologically active ingredients. The preferred composition of such particles is the same as that described for pharmacologically active ingredient-containing particles.

The ratio of opioid agonist to opioid antagonist in the pharmaceutical dosage forms according to the invention is preferably 1:1 to 3:1 by weight, for example, 2:1 by weight.

In another preferred embodiment, neither the particles nor the pharmaceutical dosage form comprise any opioid antagonist.

Preferably, the pharmaceutical dosage form according to the invention contains more than 20 wt.-%, more preferably more than 30 wt.-%, still more preferably more than 40 wt.-%, yet more preferably more than 50 wt.-%, most preferably more than 60 wt.-%, and in particular more than 70 wt.-% of compounds which are not or hardly soluble in ethanol with respect to the total weight of the pharmaceutical dosage form.

For the purpose of specification, compounds which are not or hardly soluble in ethanol have a maximum solubility in aqueous ethanol (96%) at room temperature of preferably less than 1000 mg/L, more preferably less than 800 mg/L, even more preferably less than 500 mg/L, most preferably less than 100 mg/L and in particular less than 10 mg/L or less than 1 mg/L.

Preferably, the pharmaceutical dosage form according to the invention contains more than 50 wt.-%, more preferably more than 60 wt.-%, still more preferably more than 70 wt.-%, yet more preferably more than 80 wt.-%, most preferably more than 90 wt.-%, and in particular more than 95 wt.-% of polymers which are not or hardly soluble in ethanol with respect to the overall amount of polymers contained in the pharmaceutical dosage form.

Preferred polymers which are not or hardly soluble in ethanol according to the invention are xanthan, guar gum and some types of HPMC. The skilled person knows what types of HPMC are not or hardly soluble in ethanol within the sense of the invention.

In a particularly preferred embodiment, the entire pharmaceutical dosage form according to the invention contains polymers which are not or hardly soluble in ethanol and polymers which are soluble in ethanol, wherein the amount of polymers which are not or hardly soluble in ethanol relative to the total amount of polymers contained in the dosage form is 30 to 100 wt.-%, more preferably 50 to 100 wt.-%, still more preferably 60 to 95 wt.-% or 100 wt.-%, yet more preferably 70 to 90 wt.-% or 100 wt.-%, most preferably 80 to 90 wt.-% or 90 to 100 wt.-%, and in particular more than 95 wt.-% or more than 99 wt.-%.

Preferred compositions of the pharmaceutical dosage form or, when the pharmaceutical dosage form is multiparticulate, of the particles are summarized as embodiments B¹ to B⁶ in the table here below:

| wt.-% ^{a)} | B¹ | B² | B³ | B⁴ | B⁵ | B⁶ |
|---|---|---|---|---|---|---|
| pharmacologically active ingredient | 40 ± 10 | 40 ± 5 | 35 ± 10 | 33 ± 10 | 33 ± 10 | 33 ± 10 |
| polyethylene glycol graft copolymer | 40 ± 10 | 40 ± 5 | 50 ± 15 | 57 ± 20 | 60 ± 20 | 67 ± 15 |
| additional prolonged release matrix | 10 ± 10 | 10 ± 10 | 15 ± 10 | 10 ± 7 | 7.5 ± 5 | 10 ± 10 |
| material | | | | | | |
| excipients | 10 ± 10 | 10 ± 10 | 5 ± 5 | 5 ± 5 | 5 ± 5 | 5 ± 5 |

^{a)} relative to the total weight of the dosage form and particles, respectively.

The subjects to which the pharmaceutical dosage forms according to the invention can be administered are not particularly limited. Preferably, the subjects are animals, more preferably human beings.

The pharmaceutical dosage form according to the invention or, when it is multiparticulate, the particles that contain the pharmacologically active ingredient are preferably thermoformed, preferably by melt-extrusion, although also other methods of thermoforming may be useful, such as press-molding at elevated temperature or heating of compacts that were manufactured by conventional compression in a first step and then heated above the softening temperature of the polyethylene glycol graft copolymer and the prolonged release matrix material, respectively, in a second step to form break resistant, hardened compacts, i.e. monolithic dosage forms or particles, respectively. In this regard, thermoforming preferably means the forming or molding of a mass after, before or during the application of heat. Preferably, thermoforming is performed by hot-melt extrusion.

In a preferred embodiment, the pharmaceutical dosage form according to the invention is hot-melt extruded.

In a preferred embodiment, hot-melt extrusion is performed by means of a twin-screw-extruder. Melt extrusion preferably provides a melt-extruded strand that is preferably cut into monoliths, which are then optionally compressed and formed. Preferably, compression is achieved by means of a die and a punch, preferably from a monolithic mass obtained by melt extrusion. If obtained via melt extrusion, the compressing step is preferably carried out with a monolithic mass exhibiting ambient temperature, that is, a temperature in the range from 20 to 25° C.

The strands obtained by way of extrusion can either be subjected to the compression step as such or can be cut prior to the compression step. This cutting can be performed by usual techniques, for example using rotating knives or compressed air, at elevated temperature, e.g. when the extruded stand is still warm due to hot-melt extrusion, or at ambient temperature, i.e. after the extruded strand has been allowed to cool down. When the extruded strand is still warm, singulation of the extruded strand into extruded monolithic pharmaceutical dosage forms and particles, respectively, is preferably performed by cutting the extruded strand immediately after it has exited the extrusion die.

However, when the extruded strand is cut in the cooled state, subsequent singulation of the extruded strand is preferably performed by optionally transporting the still hot extruded strand by means of conveyor belts, allowing it to cool down and to congeal, and subsequently cutting it. Alternatively, the shaping can take place as described in EP-A 240 906 by the extrudate being passed between two counter-rotating calender rolls and being shaped directly to pharmaceutical dosage forms and particles, respectively. It is of course also possible to subject the extruded strands to the compression step or to the cutting step when still warm, that is more or less immediately after the extrusion step. The extrusion is preferably carried out by means of a twin-screw extruder.

The pharmaceutical dosage forms and particles, respectively, according to the invention may be produced by different processes, the particularly preferred of which are explained in greater detail below. Several suitable processes have already been described in the prior art. In this regard it can be referred to, e.g., WO 2005/016313, WO 2005/016314, WO 2005/063214, WO 2005/102286, WO 2006/002883, WO 2006/002884, WO 2006/002886, WO 2006/082097, and WO 2006/082099.

In general, the process for the production of the particles according to the invention preferably comprises the following steps:
(a) mixing all ingredients;
(b) optionally pre-forming the mixture obtained from step (a), preferably by applying heat and/or force to the mixture obtained from step (a), the quantity of heat supplied preferably not being sufficient to heat the polyethylene glycol graft copolymer and the prolonged release matrix material, respectively, up to its softening point;
(c) hardening the mixture by applying heat and force, it being possible to supply the heat during and/or before the application of force and the quantity of heat supplied being sufficient to heat the polyethylene glycol graft copolymer and the prolonged release matrix material, respectively, at least up to its softening point; and thereafter allowing the material to cool and removing the force
(d) optionally singulating the hardened mixture;
(e) optionally shaping the particles; and
(f) optionally providing a film coating.

Heat may be supplied directly, e.g. by contact or by means of hot gas such as hot air, or with the assistance of ultrasound or microwave radiation; or is indirectly supplied by friction and/or shear. Force may be applied and/or the particles may be shaped for example by direct pharmaceutical dosage forming or with the assistance of a suitable extruder, particularly by means of a screw extruder equipped with one or two screws (single-screw-extruder and twin-screw-extruder, respectively) or by means of a planetary gear extruder.

The final shape of the pharmaceutical dosage forms and particles, respectively, may either be provided during the hardening of the mixture by applying heat and force (step (c)) or in a subsequent step (step (e)). In both cases, the mixture of all components is preferably in the plastified state, i.e. preferably, shaping is performed at a temperature at least above the softening point of the polyethylene glycol graft copolymer and the prolonged release matrix material, respectively. However, extrusion at lower temperatures, e.g. ambient temperature, is also possible and may be preferred.

Shaping can be performed, e.g., by means of a pharmaceutical dosage forming press comprising die and punches of appropriate shape.

Another aspect of the invention relates to a process for the production of a tamper-resistant, oral pharmaceutical dosage form comprising the steps of
(i) mixing a pharmacologically active ingredient, a polyethylene glycol graft copolymer and optionally further excipients; and
(ii) thermoforming the mixture obtained in step (i), wherein said mixture is simultaneously or before or after the application of heat subjected to pressure.

In a preferred embodiment, the tamper-resistant, oral pharmaceutical dosage form which is produced by said process is according to the tamper-resistant, oral pharmaceutical dosage forms described above.

A particularly preferred process for the manufacture of the particles according to the invention involves hot-melt extrusion. In this process, the pharmaceutical dosage forms and particles, respectively, according to the invention are produced by thermoforming with the assistance of an extruder, preferably without there being any observable consequent discoloration of the extrudate.

This process is characterized in that
a) all components are mixed,
b) the resultant mixture is heated in the extruder at least up to the softening point of the polyethylene glycol graft copolymer and the prolonged release matrix material, respectively, and extruded through the outlet orifice of the extruder by application of force,
c) the still plastic extrudate is singulated and formed into the pharmaceutical dosage forms and particles, respectively, or
d) the cooled and optionally reheated singulated extrudate is formed into the pharmaceutical dosage forms and particles, respectively.

Mixing of the components according to process step a) may also proceed in the extruder.

The components may also be mixed in a mixer known to the person skilled in the art. The mixer may, for example, be a roll mixer, shaking mixer, shear mixer or compulsory mixer.

The, preferably molten, mixture which has been heated in the extruder at least up to the softening point of the polyethylene glycol graft copolymer and the prolonged release matrix material, respectively, is extruded from the extruder through a die with at least one bore.

The process according to the invention requires the use of suitable extruders, preferably screw extruders. Screw extruders which are equipped with two screws (twin-screw-extruders) are particularly preferred.

In a preferred embodiment, extrusion is performed in the absence of water, i.e., no water is added. However, traces of water (e.g., caused by atmospheric humidity) may be present.

The extruded strand is preferably water-free, which preferably means that the water content of the extruded strand is preferably at most 10 wt.-%, or at most 7.5 wt.-%, or at most 5.0 wt.-%, or at most 4.0 wt.-%, or at most 3.0 wt.-%, or at most 2.0 wt.-%, more preferably at most 1.7 wt.-%, still more preferably at most 1.5 wt.-%, yet more preferably at most 1.3 wt.-%, even more preferably at most 1.0 wt.-%, most preferably at most 0.7 wt.-%, and in particular at most 0.5 wt.-%.

The extruder preferably comprises at least two temperature zones, with heating of the mixture at least up to the softening point of the polyethylene glycol graft copolymer and the prolonged release matrix material, respectively, proceeding in the first zone, which is downstream from a feed zone and optionally mixing zone. The throughput of the mixture is preferably from 1.0 kg to 15 kg/hour. In a preferred embodiment, the throughput is from 0.2 kg/hour to 3.5 kg/hour. In another preferred embodiment, the throughput is from 4 to 15 kg/hour.

In a preferred embodiment, the die head pressure is within the range of from 0.5 to 200 bar. The die head pressure can be adjusted inter alia by die geometry, temperature profile, extrusion speed, number of bores in the dies, screw configuration, first feeding steps in the extruder, and the like.

In a preferred embodiment, the die head pressure is within the range of from 2.0±1.9 bar, more preferably 2.0±1.5 bar, and in particular 2.0±1.0 bar.

The die geometry or the geometry of the bores is freely selectable. The die or the bores may accordingly exhibit a flat (film), round, oblong or oval cross-section, wherein the round cross-section preferably has a diameter of 0.1 mm to 2 mm for extruded particles and a larger diameter for extruded monolithic pharmaceutical dosage forms. Preferably, the die or the bores have a round cross-section. The casing of the extruder used according to the invention may be heated or cooled. The corresponding temperature control, i.e. heating or cooling, is so arranged that the mixture to be extruded exhibits at least an average temperature (product temperature) corresponding to the softening temperature of the prolonged release matrix material and does not rise above a temperature at which the pharmacologically active ingredient to be processed may be damaged. Preferably, the temperature of the mixture to be extruded is adjusted to below 180° C., preferably below 150° C., but at least to the softening temperature of the polyethylene glycol graft copolymer and the prolonged release matrix material, respectively. Typical extrusion temperatures are 120° C. and 150° C.

In a preferred embodiment, the extruder torque is within the range of from 30 to 95%. Extruder torque can be adjusted inter alia by die geometry, temperature profile, extrusion speed, number of bores in the dies, screw configuration, first feeding steps in the extruder, and the like.

After extrusion of the molten mixture and optional cooling of the extruded strand or extruded strands, the extrudates are preferably singulated. This singulation may preferably be performed by cutting up the extrudates by means of revolving or rotating knives, wires, blades or with the assistance of laser cutters.

Preferably, intermediate or final storage of the optionally singulated extrudate or the final shape of the pharmaceutical dosage forms and particles, respectively, according to the invention is performed under oxygen-free atmosphere which may be achieved, e.g., by means of oxygen-scavengers.

The singulated extrudate may be press-formed into pharmaceutical dosage forms and particles, respectively, in order to impart the final shape to the pharmaceutical dosage forms and particles, respectively.

The application of force in the extruder onto the at least plasticized mixture is adjusted by controlling the rotational speed of the conveying device in the extruder and the geometry thereof and by dimensioning the outlet orifice in such a manner that the pressure necessary for extruding the plasticized mixture is built up in the extruder, preferably immediately prior to extrusion. The extrusion parameters which, for each particular composition, are necessary to give rise to a pharmaceutical dosage form with desired mechanical properties, may be established by simple preliminary testing.

For example but not limiting, extrusion may be performed by means of a twin-screw-extruder type ZSE 18 or ZSE 27 (Leistritz, Nurnberg, Germany) or Thermo Scientific* Pharma 16 HME, screw diameters of 16, 18 or 27 mm. Screws having eccentric or blunt ends may be used. A heatable die with a round bore or with a multitude of bores each having a diameter of 0.2, 0.3, 0.4, 0.5, 0.6, 0.7, 0.8, 0.9, 1.0, 2.0, 3.0, 4.0, 5.0 or 6.0 mm may be used. The extrusion parameters may be adjusted e.g. to the following values: rotational speed of the screws: 120 Upm; delivery rate 0.5 kg/h for Pharma 16, 2 kg/h for a ZSE 18 or 8 kg/h for a ZSE 27; product temperature: in front of die 100 to 125° C. and behind die 125 to 135° C.; and jacket temperature: 110° C.

Preferably, extrusion is performed by means of twin-screw-extruders or planetary-gear-extruders, twin-screw extruders (co-rotating or contra-rotating) being particularly preferred.

The pharmaceutical dosage forms and particles, respectively, according to the invention are preferably produced by thermoforming with the assistance of an extruder without any observable consequent discoloration of the extrudates.

The process for the preparation of the pharmaceutical dosage forms and particles, respectively, according to the invention is preferably performed continuously. Preferably, the process involves the extrusion of a homogeneous mixture of all components. It is particularly advantageous if the thus obtained intermediate, e.g. the strand obtained by extrusion, exhibits uniform properties. Particularly desirable are uniform density, uniform distribution of the active compound, uniform mechanical properties, uniform porosity, uniform appearance of the surface, etc. Only under these circumstances the uniformity of the pharmacological properties, such as the stability of the release profile, may be ensured and the amount of rejects can be kept low.

Preferably, the pharmaceutical dosage form is multiparticulate and the particles according to the invention can be regarded as “extruded pellets”. The term “extruded pellets” has structural implications which are understood by persons skilled in the art. A person skilled in the art knows that pelletized pharmaceutical dosage forms can be prepared by a number of techniques, including:
drug layering on nonpareil sugar or microcrystalline cellulose beads,
spray drying,
spray congealing,
rotogranulation,
hot-melt extrusion,
spheronization of low melting materials, or
extrusion-spheronization of a wet mass.

Accordingly, “extruded pellets” can be obtained either by hot-melt extrusion or by extrusion-spheronization.

“Extruded pellets” can be distinguished from other types of pellets because they are structurally different. For example, drug layering on nonpareils yields multilayered pellets having a core, whereas extrusion typically yields a monolithic mass comprising a homogeneous mixture of all ingredients. Similarly, spray drying and spray congealing typically yield spheres, whereas extrusion typically yields cylindrical extrudates which can be subsequently spheronized.

The structural differences between “extruded pellets” and “agglomerated pellets” are significant because they may affect the release of active substances from the pellets and consequently result in different pharmacological profiles. Therefore, a person skilled in the pharmaceutical formulation art would not consider “extruded pellets” to be equivalent to “agglomerated pellets”.

The pharmaceutical dosage forms according to the invention may be prepared by any conventional method. Preferably, however, the pharmaceutical dosage forms are prepared by compression. Thus, particles as hereinbefore defined are preferably mixed, e.g. blended and/or granulated (e.g. wet granulated), with outer matrix material and the resulting mix (e.g. blend or granulate) is then compressed, preferably in molds, to form pharmaceutical dosage forms. It is also envisaged that the particles herein described may be incorporated into a matrix using other processes, such as by melt granulation (e.g. using fatty alcohols and/or water-soluble waxes and/or water-insoluble waxes) or high shear granulation, followed by compression.

When the pharmaceutical dosage forms according to the invention are manufactured by means of an eccentric press, the compression force is preferably within the range of from 5 to 15 kN. When the pharmaceutical dosage forms according to the invention are manufactured by means of a rotating press, the compression force is preferably within the range of from 5 to 40 kN, in certain embodiments >25 kN, in other embodiments 13 kN.

Another aspect of the invention relates to a tamper-resistant, oral pharmaceutical dosage form which is obtainable by any of the processes described above.

The pharmaceutical dosage form according to the invention is characterized by excellent storage stability. Preferably, after storage for 4 weeks at 40° C. and 75% rel. humidity, the content of pharmacologically active ingredient amounts to at least 98.0%, more preferably at least 98.5%, still more preferably at least 99.0%, yet more preferably at least 99.2%, most preferably at least 99.4% and in particular at least 99.6%, of its original content before storage. Suitable methods for measuring the content of the pharmacologically active ingredient in the pharmaceutical dosage form are known to the skilled artisan. In this regard it is referred to the Eur. Ph. or the USP, especially to reversed phase HPLC analysis. Preferably, the pharmaceutical dosage form is stored in closed, preferably sealed containers.

The pharmaceutical dosage forms according to the invention may be used in medicine, e.g. as an analgesic. The pharmaceutical dosage forms are therefore particularly suitable for the treatment or management of pain. In such pharmaceutical dosage forms, the pharmacologically active ingredient preferably is analgesically effective.

A further aspect of the invention relates to the pharmaceutical dosage form as described above for use in the treatment of pain.

A further aspect of the invention relates to the use of the pharmacologically active ingredient for the manufacture of a pharmaceutical dosage form as described above for treating pain.

A further aspect of the invention relates to a method of treating pain comprising the administration of the pharmaceutical dosage form as described above to a subject in need thereof.

A further aspect according to the invention relates to the use of a pharmaceutical dosage form as described above for providing prolonged release of the pharmacologically active ingredient contained therein.

A further aspect according to the invention relates to the use of a pharmaceutical dosage form as described above for avoiding or hindering the abuse of the pharmacologically active ingredient contained therein.

A further aspect according to the invention relates to the use of a pharmaceutical dosage form as described above for avoiding or hindering the unintentional overdose of the pharmacologically active ingredient contained therein.

In this regard, the invention also relates to the use of a pharmaceutical dosage form as described above for the prophylaxis and/or the treatment of a disorder, thereby preventing an overdose of the pharmacologically active ingredient, particularly due to comminution of the pharmaceutical dosage form by mechanical action.

In a particularly preferred embodiment,
the pharmaceutical dosage form according to the invention is monolithic or multiparticulate or a MUPS formulation; and/or
the pharmaceutical dosage form according to the invention is hot-melt extruded; and/or
the pharmaceutical dosage form according to the invention provides prolonged release of the pharmacologically active ingredient having psychotropic action; and/or
the pharmacologically active ingredient having psychotropic action is an opioid or a physiologically acceptable salt thereof; and/or
the content of the pharmacologically active ingredient is within the range of from 1 to 35 wt.-%, based on the total weight of the pharmaceutical dosage form; and/or
the polyethylene glycol graft copolymer comprises repetition units derived from ethylene glycol, and vinyl acetate and vinyl caprolactam; and/or
the polyethylene glycol graft copolymer contains 60±30 wt.-%, more preferably 60±20 wt.-% of ethylene glycol repetition units, relative to the total weight of the polyethylene glycol graft copolymer; and/or
the content of the polyethylene glycol graft copolymer is within the range of from 45 to 70 wt.-%, relative to the total weight of the pharmaceutical dosage form or, when the pharmaceutical dosage form is multiparticulate, relative to the total weight of the particles that contain the pharmacologically active ingredient; and/or
the pharmacologically active ingredient is embedded in a prolonged release matrix containing the polyethylene glycol graft copolymer as prolonged release matrix material and additional prolonged release matrix material; wherein
   the content of the additional prolonged release matrix material is in the range of 5 to 30 wt.-%, relative to the total weight of the prolonged release matrix; and/or
   the additional prolonged release matrix material is a cellulose ether, preferably a cellulose ether having a weight average molecular weight of at least 50,000 g/mol.

The following example further illustrates the invention but are not to be construed as limiting its scope.

### EXAMPLE

The following compositions were prepared:

| Batch | |
|---|---|
| comparative | inventive |

| Excipient | mg | wt.-% | mg | wt.-% |
|---|---|---|---|---|
| Tramadol-HCl | 80.0 | 13.3 | 80.0 | 13.3 |
| Polyethylenoxide | 370.0 | 61.7 | — | — |
| 7,000,000 | | | | |
| Soluplus ® | — | — | 370.0 | 61.7 |
| Hypromellose 100,000 | 60.0 | 10.0 | 150.0 | 25.0 |
| Macrogol 6,000 | 90.0 | 15.0 | — | — |
| Totals | 600.0 | — | 600.0 | — |

Hot-melt extrusion was performed on a Leistritz ZSE27 Micro extruder using the following instrument settings:

| Screw configuration | “Medium shear” | |
|---|---|---|
| Temperature | up to 135° C. | |
| Screw speed | 100 | rpm |
| Die diameter (mm) | 10 | mm |
| Throughput | 3.5 | kg/h |

The extrudate strand was cooled in a Sollich cooling tunnel to a cutting temperature of 45° C. The batch could be manufactured under standard process conditions. However, the strand could not be cut using the cutting machine as the blade was deformed during cutting. Cutting was therefore performed manually using a food slice. During the extrusion a very low melt pressure (˜2 bar) together with a high torque (92-102%) was observed.

Tablet shaping was performed manually on a Korsch EK0 equipped with round punches with a diameter of 12 mm and a curvature of 9 mm without embossing.

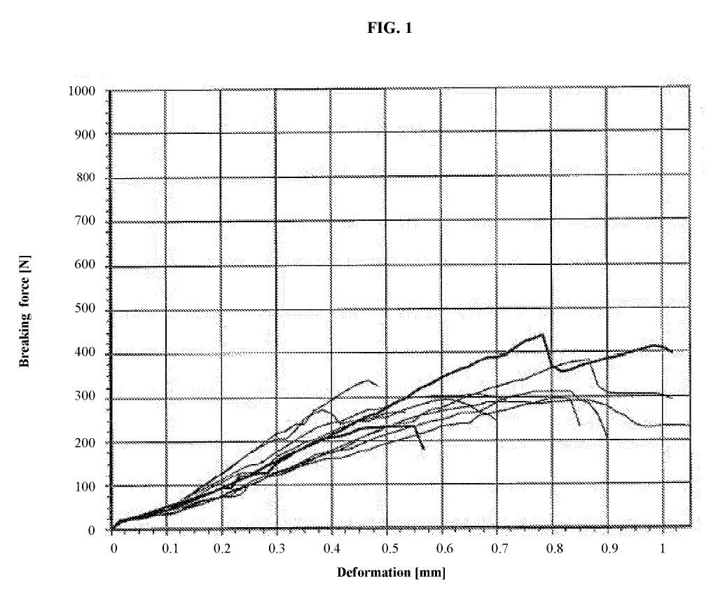

The result of the breaking strength test is shown in the load-displacement diagram of FIG. 1. The tablets were brittle and had a mean breaking force of below 350 N.

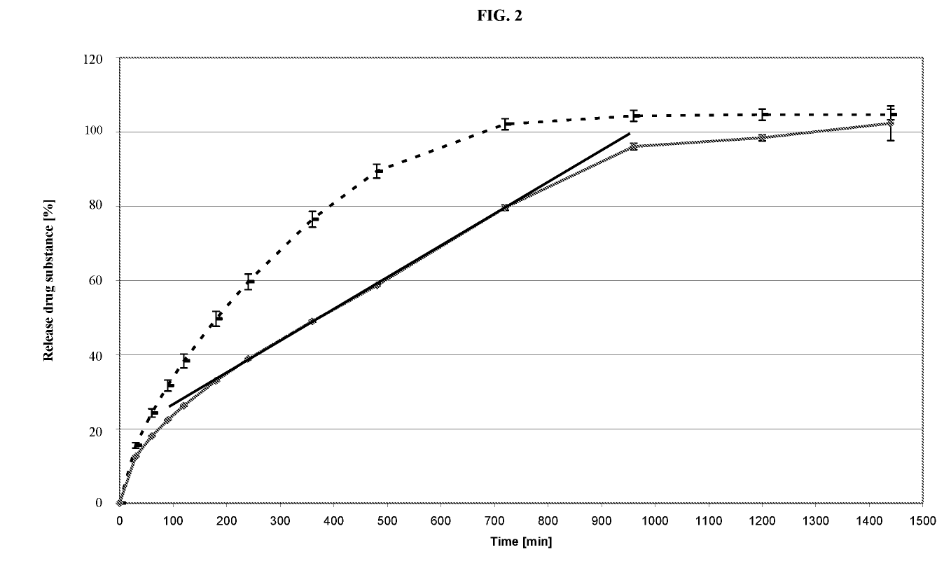

The results of the in vitro dissolution test are shown in FIG. 2 (Dissolution profiles of Tramadol-HCl 80 mg TRF prolonged release tablets; comparative example, inventive example; 37° C., rpm=75, 600 mL phosphate buffer, pH=6.8, n=3±s). It becomes clear from FIG. 2 that the batch containing Soluplus® instead of polyethyleneoxide shows a slower release rate and between 30% and 90% released drug amount a linear dissolution behavior (zero order kinetics).

1. A tamper-resistant, oral pharmaceutical dosage form comprising a pharmacologically active ingredient having psychotropic action and a polyethylene glycol graft copolymer, wherein the polyethylene glycol graft copolymer is present in the pharmaceutical dosage form in a content of at least 25 wt.-%, relative to a total weight of the pharmaceutical dosage form.

2. The pharmaceutical dosage form according to claim 1, wherein
(i) the pharmacologically active ingredient is embedded in a prolonged release matrix comprising the polyethylene glycol graft copolymer; and/or
(ii) the pharmaceutical dosage form provides prolonged release of the pharmacologically active ingredient.

3. The pharmaceutical dosage form according to claim 1, wherein the polyethylene glycol graft copolymer comprises repetition units derived from ethylene glycol, vinyl acetate and vinyl caprolactam.

4. The pharmaceutical dosage form according to claim 1, wherein the polyethylene glycol graft copolymer has the general formula (I) 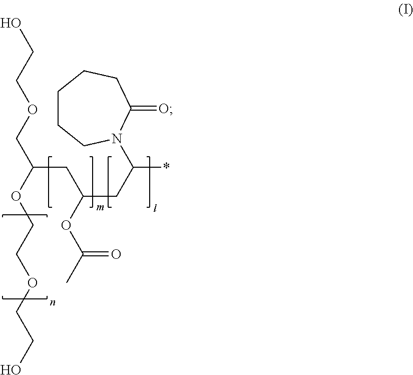
wherein
index l is 10 to 10,000;
index m is 20 to 20,000; and
index n is 30 to 30,000.

5. The pharmaceutical dosage form according to claim 4, wherein.
index l is 500 to 900;
index m is 800 to 1500; and
index n is 2000 to 3000.

6. The pharmaceutical dosage form according to claim 1, wherein the polyethylene glycol graft copolymer has an average molecular weight of from 90,000 g/mol to 140,000 g/mmol.

7. The pharmaceutical dosage form according to claim 1, wherein the content of the polyethylene glycol graft copolymer is within the range of from 25 to 80 wt.-%, relative to the total weight of the pharmaceutical dosage form.

8. The pharmaceutical dosage form according to claim 7, wherein the content of the polyethylene glycol graft copolymer is at least 30 wt.-%, relative to the total weight of the pharmaceutical dosage form.

9. The pharmaceutical dosage form according to claim 1, which is monolithic and has a breaking strength of at least 200 N; or which is multiparticulate comprising a plurality of individual particles, wherein at least a fraction of the individual particles have a breaking strength of at least 200 N.

10. The pharmaceutical dosage form according to claim 1, which is monolithic and has an extension in any direction of at least 2.0 mm; or which is multiparticulate comprising a plurality of individual drug-containing particles, wherein the individual drug-containing particles have an extension in any direction of at least 2.0 mm.

11. The pharmaceutical dosage form according to claim 1, wherein the pharmacologically active ingredient is an opioid or a physiologically acceptable salt thereof.

12. The pharmaceutical dosage form according to claim 1, which is hot-melt extruded.

13. A process for the production of a tamper-resistant, oral pharmaceutical dosage form comprising the steps of:
(i) mixing a pharmacologically active ingredient, a polyethylene glycol graft copolymer and optionally further excipients; and
(ii) thermoforming the mixture obtained in step (i), wherein said mixture is simultaneously subjected to both heat and pressure or subjected to pressure either before or after the application of heat.

14. The process according to claim 13, wherein the tamper-resistant pharmaceutical dosage form comprises a pharmacologically active ingredient having psychotropic action and the polyethylene glycol graft copolymer, wherein the polyethylene glycol graft copolymer is present in the pharmaceutical dosage form in a content of at least 25 wt.-%, relative to a total weight of the pharmaceutical dosage form.

15. A tamper-resistant, oral pharmaceutical dosage form obtainable by the process according to claim 13.

16. A tamper-resistant, oral pharmaceutical dosage form obtainable by the process according to claim 14.

17. A method of treating pain comprising administering to a patient in need thereof a dosage form according to claim 1.

18. A method of treating pain comprising administering to a patient in need thereof a dosage form according to claim 13.
